SEQUENCE LISTING (16 sequences)

SEQ ID NO: 1 - protein, 441 aa, Artificial Sequence
  Description of Artificial Sequence/note = Synthetic Construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 410 aa, Artificial Sequence
  Description of Artificial Sequence/note = Synthetic Construct
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 695 aa, Artificial Sequence
  Description of Artificial Sequence/note = Synthetic Construct
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVRVPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 4 - protein, 770 aa, Artificial Sequence
  Description of Artificial Sequence; note = synthetic construct
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - protein, 467 aa, Artificial Sequence
  Description of Artificial Sequence/note = Synthetic Construct
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 6 - protein, 448 aa, Artificial Sequence
  Description of Artificial Sequence/note = Synthetic Construct
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTTFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 7 - protein, 42 aa, Artificial Sequence [sequence omitted]

SEQ ID NO: 8 - DNA, 2833 bp, Artificial Sequence
  Description of Artificial Sequence/note = Synthetic Construct
ctttctattcctaaagaccttgggtgaccaaaatcttattttaaaaaataaaactgtttattaaaacttttttgtttcaaagaaccatatgtatagtgaaatttataaaaatatcaatttttaaaaagctggtgtactcatttatgttatgaactctaaaaccatatactgactgcaagtgatgatgtatagagtgatgtttacgagtaaacatatttagttgtatacatcctactgagcacattttgatgtatgaaataacattacaagctttatccaaattaagccattttaaaacactgccaattgaaaatacaaatcctggaaaaaatcgtctttagcgcagtcatttgagccatcctaatccgttacctcagaccataataagaagggataacactagctgtagcaatggaacacatctgtttcacacaatcatatctcctgcgccggtgctaagcagattcagcgtgatcataacatgctttccactcataaatgtaaatttacaatttgcacatgtaaaacagacacttttgagatattggataaaaaaacaagagtatattgcttagtttcatccaccagtcatccccacagcgtttggaaggccataaaaagtgtctaaaatcaatgatcattgaaagagcacaagagagactcttacgctgtaatgccactggggacaaaagtgacagtctcttaatgggctcttctggaggggctcctgaacattaaaaattatcagcgaaattaccgaaagagcttcaagcaactggcatgcttgatcctctgcgtcggggcggtgaataggtgcttcagatgccctcttacccacgggctggattcagctgccccgctaccagcggagaccccctaatgagcctctgcaattaagtttattcatgttaagtgtgaacggggtgcgtgcggaactgtgggcagctaacagacctgggttctttgtgccacaagtgctgcctttattcggctcacaaagcagaaaacaacacccgcacctataatggcgccctcggctgggtctaagaaacgtggcgagttgacagagcagagtgggcggggttaagacagactgacagcgggacccatctccatcctcttattaacgcttaacgagtgccttccccatgcaatattcatcgccactaatatcatccaagctctgagctgagctggccacttatgtaaggcaattatgtaaaatatcagacagggcccacactcagaatctgactggggtagagacgcgggacgagaaccgagagcaagaactgaaagtgaaagtgaccactaaagggaggagaggacagaggggcaggatgtgtcaagattaccagagaacacttggccagaaatgcgcaaccattggagctctccggattacccaaaggttaacgagtttgaacgcctctgcccactcgcccatctctgatggtttcccaagaactcctcaagcaaaatatatataattgtgtgtattatgcacagacacgagaaaatgctgtttttctgatctgcattacagcacatttgcccgccaacgacaataccacccactcggtacctcgctgactcctgatgcctgatacctgcgcggtgactgtctacaatctgcataatcaagagaagttgtgttgaagacgagcgccacacaaccgtttccacaaggtcacccaaggccggtgcagatgtaggtgaggtctccataaacagactgaaataaacacatcctccgctgggaacaacaaccccctcacgcctcatgcatttccataagcctacatgcatctcttccaacttatggagactcgcacctaccaacatccgcacaacaaagatatacagagcgcgctccctcaggtcaaggccctgtgggggtctgtgcagaaataggtcatttgtcacacatcaagtcctggggcaggagatgcattatagatgagaccaaacagcctgtctcggtgagctctacccactccctgagactagaaatgggggaagggagcttgagataacaaccgctgcaatcactgtgtcgatgtttaatatcagcaccaaccggaacaataagagatgggtgcattcatgttcacatcttaccagtcaagtatcatcgaaccggcttgataaccacacctcgtgtaatagctgagcagatagttgtcattttaaagcgttggcctttgtcgattatgtaatgcgcacattcaacacatggtaatatagaaacggttatgtcgaggttgttttgtccagagatgaccttcacacagttacagccgctctgcatccacacaaatggaggacttaatcgtggactgcattcttagaaatgatctacaaagacaaataatgtgaaatcaagaaaggacaaaatttaagtaaggggatgagggagagagagaacgaggggcaaggagaaagcatggctcctgtctttttctgcacccatctgttcggagtgcaggtggagctctattcactcagctctgcatgtgtgtttggggggggcaggaagaaagggagggcaaaaggaagagtggagagatggtgggggctggagggatggggggttctcggtgatctctcctgaaggggataatgggagagcagcgctttgcaatggctgccatgtagtaccctcccctgcacaattagccaatcagcagcaagctctgccagccagaaggacacataaaagaagaacattgcagcagaggcacagaaggagcctgcgaggagctgggaaatacacacacaacagcagaaccacaacaccctcccctggacacaccctactggggatcactgc

SEQ ID NO: 9 - DNA, 54 bp, Artificial Sequence
  Description of Artificial Sequence/note = Synthetic Construct
atgcataaggttttgctggcactgttctttatctttctggcaccagcaggtacc

SEQ ID NO: 10 - DNA, 4808 bp, Artificial Sequence
  Description of Artificial Sequence/note = Synthetic Construct
atattttgggttatggctaaaataattaatgtctaaaacgggattacgcgtttttcgtaaagctcaaagacgcatgtgccaaaaatagccttttattaaattgtttggttattaaaatattattcaacttattttacatccatggaaagagacatggcctcttctatttgacctgcatgtgttaaaacgaaatgccaaaataaagaaaaaaatgtaattcaacatgtaaggctattcaaaaacaatacacaggtacaaaacatatctttgttaatgaaactaatttacagtttgtttattaaaacacactataaatgccatagaacattttggagatgcatgcgttatacattgcgtgatttaacagatcaattaaagtcgtattttgcgccagcatttcaatgggcataacgacttaatgttttcctctagaatgattacaaatgtgaaagcgaatgtgatgtgattgagttgaagaattagttttttttggaatgccccaaggacgcatgcattagcccacctgtgctgtttatttaaatcattgactccaagagctgtcagccacaaaaggagggcgggcgcgctgtcatcacccatcagatttatgactgccacacaatcattttccgactaaactaacgccatcatcactcagaacaagaacttcatgagtcgcacaagacaagttataataaatgcattacagcgaatgcatgcacaaacgcgagaaccacttttgctgcaaaataatgtggattgttggttgaaatgaaaactgggtgagatgcttttctttcaatccctgttatccatgcttcagcagaggacaggaggcttgtgactttgcctgtgcctgtgtctgcccccgagtgccctgtcacaatctaattacccgtgagtaaaggacaataccgcttcagctggtctgtgtcattccccctatatcccagtgcctgcttattttcacaaacccttctgcgccgctttctgccccctcctgccctcttttaaccccacggagaatgataaatgcgcggtgagggaacgaacgggcaaagccatttcacggcacctgttaattaagggaatgattgcctccatttttcgctgagctcgtttccagcgtgctccattatttgtgatgcgattaattgaaagcgaatgtgacatcacaacgaacgtgatgtcattgtcgccgtcacacagtagaacgacagagttacataagaaataaagtctgcatgcatacatttatgcatggcgttttaaagaagagcgcacactgggttagagtcctcggtggggtcagccacttcggtaacaccccaagcattcaatgctaagcccttaaaaggacagcgtcttttgttctaacatcgagagcaccgggattaccacaggtatttagttcaggtattctctaagaatatttagccctaggtgagctgaaccaagagcagtcattagcgctaaaactggctctgatgggaagggctaacacacacacacacacacacacacacacacacacacacattataataaatgtaatgtcatgtttacaacaactccggcagtgatgctgcatattggcggcgtacatacactaaatgttttaatgtagtctgtaagactagagaatcagaaattaatttacacagaaattacaaaaataaatacatgtttaaatagttaataaacataattcaaatatgtaatgtattatcgtgtattttaacattaatggatgaggtggttcaaatgcattttgcacaaaataaaatcgaagcagcttcaaatcgtaaagataatagtcggtagcattgaatctgctttaacatttacttttagcgaaggctactttattaaggaagctcatattaactcccaatgaatgtctgctattgcacctttttgaggtgtagactgtgtaaaatgcatcactgcacagcaaaatcaagcgtcatattatcctgtacattctaatttgttggcttcaggctgccagggctctttgtgctgtgtagggcccctggccagattccagtgtgttaaaaagggatttacgcatctgatattgtcacacaataaggacaaatagcccgtttgagcatctttatacaaccaacgctgacagaggttctgcggtttaagtgcttagtgttgcatttgtgcttaaattgattgtttggtgttcaaccctcactggaaaaaaatcttttgatgcaaatgggtgcgtttagataaaaagaagcaaagcctagaactaaagcctagaatttatattgcactgtagatgtggatggttatgggaaagttttttgagatactgtggggcgagtcacggcgtcagagtggcggccggtaggggctctaaactcgcgctccaattattgcctgtcagtcatcatcgctttagattagagcatgcggattaaaactcatgcctttaaataataacaacagcgtcaatattatcaaaaagacacatcacgcttatttaaaatctacgaaatgtgttaaagcataatttgtactactggttgattgttgtagacctgaaatcctgtcagatagaaatgaactacccggaccactggtagttaagtctctcttgtgttatctttgattgatccaaccagacaagctagttaaattaataatttataagcgcaaagcgttggtacaagcagttagagggagaaaggtgagaagaagcaatacaaagtagctaaattcacaatgcattacattgtccattttagaaatgaaacacgaggatttaatgttaaatgaatacagagtagctataatcagcaatacaaagtagctaaattcagcaatacaaagtagctaaattcagcaatacaaagtagctatattcagcaatacaaagtagctaaattcagcaatacaaagtagctatattcagcaatacaaagtagctatattcagcaatacaaagtagctaaattcagcaatacaacgtagctatactttgtagctatacactgtatccattttagaaatgcacacgatgattttctgttaaaaatcactgctcatttgaattagattatttgaattggagcttacattgcatgtaattagtaagcaaattcggcttaacaaatttgaaacgcgtttttttttctcgactaaattaattaagaaaatgtattattgatgggtgcaaacagtaacaatttattaaaccctctatgcaaatgaggtgttcagctgactaacctgcatccacagtttatctaaacgcttatcaaactaattggcgacgttctgtctttctgcctgcggtgggcgagcctgctgcttgttttgccacgagataattgtacgcaagaatcaacgaagctgccctaatggccaccaattggctttatttggacctgcccatgcgacctgtcggcacctccaagagacgggctcgctattaatatgtaaagtgacgtttgatcgcttgaaacggcatacaaagacagtgttttcacaagaagaatgtggtgacaactcatttaaaactattagacgcgcaagaacaatagcccccaatttagagaccataaaatactcctccccaattaatgcctgaggtgctaggagttgagtttgcttgcattaggcacatatctcatgtgacacttcagtgttacaggttttgttgttttaagctaatgttaatggtcagggaacagctcgtaatcacaatatatatttaaaacaaatgattattatgaatgcaataggccaaatcgatattcattaatagaatagaggcattttaatacatttctgcacaattaaaaattaaatataatcctgcaagtctataattatattattcacatcatttaatgtcctaaaaataaatttaaaaaatagcattaggctgcaacttagattttaggcttttctgttagcacttgagtaaaaagacatcattacacaccatcaacgtgaagctctaaaaagggtaaaaagatctcaataaattgctgcgctgaatgatgagtctctcagctctctggatgtggagcagtaggccgacagtcgccgtggcatttcggaaagcatgctgtccgagccaatggcagtcagcgcgctctgctattggttcccagggcgctcactgccagctcgtgtccccgcccatgttcgtaagatatggaatctactggcgccagttccgacagtacacaggcacaattcattaatgagacttctctccgctttagacagacgcagagttttagggagactttaacaatcgggctgtggacaatttaaaccagtggcgaattacgaacgtcaacaggcatcttgaggattaacattctttgcgcaggactaacacgggaaaaataaacgcaggattggagtgctgaaatgcaactttgcgccgtgagtacttcccgatagttatttgaaattgcgagcatttaattgagcgatttaattgattgactacaaaagttagcctacttatattaactgaggcgtcgtcgtgtgaattaagatctgtcttgcactgtgtttaacgtcaacactgagatgcttctatctgttattctcttacaggtgtccctggccacccttgaatgcaaagaagcaggacctctacactccttcaaaaataaaagcatgctcagaaagtaaacagagcatcgccacctgaagcattaagctaacgacagatattttaataatctaacggactatagtggtgctttcgggtctgtagtgtcaagtaaacttttccaagcattttctaagcgcggacacttgag

SEQ ID NO: 11 - DNA, 1116 bp, Artificial Sequence
  Description of Artificial Sequence/note = Synthetic Construct
tattttgggttatggctaaaataattaatgtctaaaacgggattacgcgtttttcgtaaagctcaaagacgcatgtgccaaaaatagccttttattaaattgtttggttattaaaatattattcaacttattttacatccatggaaagagacatggcctcttctatttgacctgcatgtgttaaaacgaaatgccaaaataaagaaaaaaatgtaattcaacatgtaaggctattcaaaaacaatacacaggtacaaaacatatctttgttaatgaaactaatttacagtttgtttattaaaacacactataaatgccatagaacattttggagatgcatgcgttatacattgcgtgatttaacagatcaattaaagtcgtattttgcgccagcatttcaatgggcataacgacttaatgttttcctctagaatgattacaaatgtgaaagcgaatgtgatgtgattgagttgaagaattagttttttttggaatgccccaaggacgcatgcattagcccacctgtgctgtttatttaaatcattgactccaagagctgtcagccacaaaaggagggcgggcgcgctgtcatcacccatcagatttatgactgccacacaatcattttccgactaaactaacgccatcatcactcagaacaagaacttcatgagtcgcacaagacaagttataataaatgcattacagcgaatgcatgcacaaacgcgagaaccacttttgctgcaaaataatgtggattgttggttgaaatgaaaactgggtgagatgcttttctttcaatccctgttatccatgcttcagcagaggacaggaggcttgtgactttgcctgtgcctgtgtctgcccccgagtgccctgtcacaatctaattacccgtgagtaaaggacaataccgcttcagctggtctgtgtcattccccctatatcccagtgcctgcttattttcacaaacccttctgcgccgctttctgccccctcctgccctcttttaaccccacggagaatgataaatgcgcggtgagggaacgaacgggcaaagccatttcacggcacctgttaattaagggaatgattgcctccatttttcgctgagct

SEQ ID NO: 12 - DNA, 1326 bp, Artificial Sequence
  Description of Artificial Sequence; note = synthetic construct
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtga

SEQ ID NO: 13 - DNA, 2088 bp, Artificial Sequence
  Description of Artificial Sequence; note = synthetic construct
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 14 - DNA, 2313 bp, Artificial Sequence
  Description of Artificial Sequence; note = synthetic construct
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactag

SEQ ID NO: 15 - DNA, 1404 bp, Artificial Sequence
  Description of Artificial Sequence; note = synthetic construct
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctag

SEQ ID NO: 16 - DNA, 1347 bp, Artificial Sequence
  Description of Artificial Sequence; note = synthetic construct
atgctcacattcatggcctctgacagcgaggaagaagtgtgtgatgagcggacgtccctaatgtcggccgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgacattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

(19) United States
(12) Patent Application Publication
(10) Pub. No.: US 2008/0201786 A1
(43) Pub. Date: Aug. 21, 2008

(54) Transgenic Zebrafish Models of Alzheimer's Disease

(75) Inventor: Amy L. Rubinstein, Atlanta, GA (US)

Correspondence Address: NEEDLE & ROSENBERG, P.C., SUITE 1000, 999 PEACHTREE STREET, ATLANTA, GA, 30309-3915, US

(73) Assignee: ZYGOGEN, LLP, Atlanta, GA (US)

(21) Appl. No.: 11/883,197

(22) PCT Filed: Jan. 27, 2006
(86) PCT No.: PCT/US06/03165

§ 371 Date: Nov. 1, 2007

Related U.S. Application Data
(60) Provisional application No. 60/647,493, filed on Jan. 27, 2005.

(51) Int. Cl. G01N 33/50; A01K 67/027

(52) U.S. Cl. 800/3; 800/20

(57) ABSTRACT

The present invention relates to zebrafish models for Alzheimer's disease that allow recapitulation of pathologies associated with Alzheimer's disease. This invention also relates to methods for screening of compounds for their ability to modulate a pathology associated with Alzheimer's disease in vivo in a whole vertebrate organism. The present invention further relates to methods of identifying gene targets for compounds that modulate a pathology associated with Alzheimer's disease.

This application claims the benefit of U.S. Provisional Application No. 60/647,493 filed Jan. 27, 2005, which is hereby incorporated herein by reference in its entirety.

### FIELD OF THE INVENTION

The present invention relates to zebrafish models for Alzheimer's disease that allow recapitulation of pathologies associated with Alzheimer's disease. This invention also relates to methods for identifying compounds that modulate a pathology associated with Alzheimer's disease in vivo in a whole vertebrate organism. The present invention further relates to methods of identifying gene targets for compounds that modulate a pathology associated with Alzheimer's disease.

### BACKGROUND

Alzheimer's disease (AD) is characterized by accumulation of neuritic plaques and neurofibrillary tangles in the brain with subsequent neuronal cell death, resulting in progressive cognitive decline. Current drugs in this therapeutic area treat only the symptoms and do nothing to stop the progression of the disease. As the population ages, an increasing number of people are diagnosed with this devastating disease. It is clear that new approaches are required to identify drugs that can protect neurons from the onslaught of AD.

Several proteins have been implicated in AD pathology, including those that are components of plaques and tangles such as Amyloid beta (Aβ) and Tau. Mutations in the Amyloid precursor protein (APP), Apolipoprotein E (apoE) and Presenilins 1 and 2 have all been linked to familial forms of AD in humans.

Mutations in Tau have been linked to frontotemporal dementia and parkinsonism linked to chromosome 17 (FTDP-17), a condition characterized by Tau inclusions similar to those observed in AD brains (Hutton et al., 1998). Mutant Tau has been shown to form neurofibrillary aggregates more readily than wild-type Tau. Alternative splicing of tau results in several Tau isoforms in adult humans. For example, alternative splicing of exon 10 results in proteins with 3 or 4 C-terminal repeats. Isoforms with 4 C-terminal repeats polymerize microtubules more efficiently and have been shown to aggregate more readily than the 3 repeat form (reviewed in Buce et al., 2000). Overexpression of human Tau in both Drosophila and C. elegans have also been shown to cause neurological dysfunction (Wittmann et al., 2001; Kraemer et al., 2003)

APP is processed by secretases in three locations (Racchi and Govoni, 2003). The action of the beta secretase, beta site APP cleaving enzyme 1 (BACE1), and gamma secretases (possibly the presenilins) result in Aβ peptides, varying in length from 39 to 43 amino acids. The longer 42-43 amino acid species tend to aggregate more readily and are more abundant in amyloid plaques of AD patients (reviewed in Verdile et al., 2004). However, the correlation between amyloid plaques and neuronal cell death is not clear and recently, a role for soluble Aβ species in neurodegeneration has been postulated (Klein et al., 2001). Numerous mutations in APP, including some that reside within the Aβ peptide, have been linked to familial forms of AD. Mutations in both Presenilin-1 and Presenilin-2, which have been shown to be involved in gamma secretase cleavage of APP, have also been correlated with familial forms of AD (reviewed in Tandon and Fraser, 2002).

Several mouse models of AD have been developed by overexpressing mutant forms of APP under the control of neuron-specific promoters (reviewed in Guenette et al., 1999). In addition, overexpression of the human Aβ peptide resulted in muscle-specific aggregates in C. elegans (Link, 1995).

AD is a top priority for most major pharmaceutical companies. AD affects over 4 million Americans each year and the incidence is increasing as the average age of the US population rises. It is important to note, however, that AD is not a normal part of aging. In addition to the loss of life and reduced quality of life, the economic cost to society is enormous given that the average AD patient lives 8-10 years following diagnosis and these patients require high levels of care to get through their day. Therefore, it is clear that new therapeutics must be developed to treat this disease.

### BRIEF DESCRIPTION OF THE FIGURES

FIG. 1 shows that overexpression of Tau-AcGFP fusion proteins under the control of the elav promoter causes reduction in fluorescence in the brain of zebrafish embryos expressing red fluorescent protein in neurons. Panels A, B, C and D are bright field images of 5 days post fertilization transgenic larvae that express dsRedExpress specifically in neurons. Panels E, F, G and H are fluorescence images. Panels A and E are control larvae injected with vehicle alone. Panels B and F show larvae injected with a construct encoding a wild type Tau isoform with 3 microtubule binding domains fused to AcGFP. Panels C and G show larvae injected with a construct encoding a wild type Tau isoform with 4 microtubule binding domains fused to AcGFP. Panels D and H show larvae injected with a construct encoding the Tau-P301L mutant isoform fused to -AcGFP.

### SUMMARY OF THE INVENTION

The present invention provides zebrafish that allow recapitulation of pathologies associated with Alzheimer's disease. This invention also provides methods of identifying compounds that modulate a pathology associated with Alzheimer's disease in vivo in a whole vertebrate organism. The present invention further provides methods of identifying gene targets for compounds that modulate a pathology associated with Alzheimer's disease.

### DETAILED DESCRIPTION OF THE INVENTION

The present invention may be understood more readily by reference to the following detailed description of the preferred embodiments of the invention and the Example included therein.

Before the present compounds and methods are disclosed and described, it is to be understood that this invention is not limited to specific proteins or specific methods. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise.

“Optional” or “optionally” means that the subsequently described event or circumstance may or may not occur, and that the description includes instances where said event or circumstance occurs and instances where it does not.

The present invention is more particularly described in the following examples which are intended as illustrative only since numerous modifications and variations therein will be apparent to those skilled in the art.

The zebrafish has become a popular model for disease model development and drug discovery (reviewed in Rubinstein, 2003). Zebrafish embryos are produced in large numbers, develop outside the mother and are transparent, facilitating the observation of tissues and organs, including neurons. The overall conservation of physiology and gene function combined with the advantages of the zebrafish system make AD models in zebrafish attractive alternatives to current approaches.

The present invention provides zebrafish that express one or more proteins associated with Alzheimer's disease in order to mimic or recapitulate one or more pathologies associated with Alzheimer's disease. The zebrafish of the present invention can also overexpress one or more proteins involved in Alzheimer's disease in order to mimic or recapitulate one or more pathologies associated with Alzheimer's disease. By “overexpress” is meant an increase in expression of a protein associated with Alzheimer's disease as compared to expression of the protein in a wildtype zebrafish that does not exhibit a pathology of Alzheimer's disease. The present invention also provides methods of utilizing these zebrafish to identify compounds and/or gene targets that can be utilized to treat Alzheimer's disease. As utilized throughout, Tau, APP, amyloid is, apoE, Presenilin 1, Presenilin 2 and fragments thereof are considered proteins associated with Alzheimer's disease. Mutant versions of these proteins and fragments of the mutant versions of the proteins are also considered proteins associated with Alzheimer's disease.

The zebrafish of the present invention, including zebrafish cells and zebrafish embryos, can be transgenic or non-transgenic. The transgenic zebrafish of this invention can be a transient or a stable transgenic zebrafish. As used herein, transgenic zebrafish refers to zebrafish, or progeny of zebrafish into which an exogenous construct has been introduced. A zebrafish into which a construct has been introduced includes fish which have developed from embryonic cells into which the construct has been introduced. As utilized herein, an exogenous construct is a nucleic acid that is artificially introduced or was originally artificially introduced into an animal. The term artificial introduction is intended to exclude introduction of a construct through normal reproduction or genetic crosses. That is, the original introduction of a gene or trait into a line or strain of animal by cross breeding is intended to be excluded. However, fish produced by transfer, through normal breeding, of an exogenous construct (that is, a construct that was originally artificially introduced) from a fish containing the construct are considered to contain an exogenous construct. Such fish are progeny of fish into which the exogenous construct has been introduced. As used herein, progeny of a fish are any fish which are descended from the fish by sexual reproduction or cloning, and from which genetic material has been inherited. In this context, cloning refers to production of a genetically identical fish from DNA, a cell, or cells of the fish. The fish from which another fish is descended is referred to as a progenitor fish. As used herein, development of a fish from a cell or cells (embryonic cells, for example), or development of a cell or cells into a fish, refers to the developmental process by which fertilized egg cells or embryonic cells (and their progeny) grow, divide, and differentiate to form an adult fish.

The present invention provides a transgenic zebrafish that expresses a Tau polypeptide, comprising a zebrafish neuron specific expression sequence operably linked to a sequence encoding a Tau polypeptide, wherein the Tau polypeptide is expressed in the neurons of the transgenic zebrafish and wherein the transgenic zebrafish exhibits a pathology associated with Alzheimer's disease.

Further provided by the present invention is a transgenic zebrafish that expresses an amyloid precursor protein (APP) polypeptide, comprising a zebrafish neuron specific expression sequence operably linked to a sequence encoding an APP polypeptide, wherein the APP polypeptide is expressed in the neurons of the transgenic zebrafish and wherein the transgenic zebrafish exhibits a pathology associated with Alzheimer's disease.

Also provided by the present invention is a transgenic zebrafish that expresses an amyloid β polypeptide, comprising a zebrafish neuron specific expression sequence operably linked to a sequence encoding an amyloid β polypeptide, wherein the amyloid β polypeptide is expressed in the neurons of the transgenic zebrafish and wherein the transgenic zebrafish exhibits a pathology associated with Alzheimer's disease.

Also provided by the present invention is a transgenic zebrafish that expresses an apolipoprotein E (apoE) polypeptide, comprising a zebrafish neuron specific expression sequence operably linked to a sequence encoding an apoE polypeptide, wherein the apoE polypeptide is expressed in the neurons of the transgenic zebrafish and wherein the transgenic zebrafish exhibits a pathology associated with Alzheimer's disease.

Also provided by the present invention is a transgenic zebrafish that expresses a presenilin 1 polypeptide, comprising a zebrafish neuron specific expression sequence operably linked to a sequence encoding a presenilin 1 polypeptide, wherein the presenilin 1 polypeptide is expressed in the neurons of the transgenic zebrafish and wherein the transgenic zebrafish exhibits a pathology associated with Alzheimer's disease.

Also provided by the present invention is a transgenic zebrafish that expresses a presenilin 2 polypeptide, comprising a zebrafish neuron specific expression sequence operably linked to a sequence encoding a presenilin 2 polypeptide, wherein the presenilin 2 polypeptide is expressed in the neurons of the transgenic zebrafish and wherein the transgenic zebrafish exhibits a pathology associated with Alzheimer's disease.

Also provided by the present invention is a transgenic zebrafish that expresses one or more of the proteins selected from the group consisting of: Tau, APP, amyloid β, apoE, Presenilin 1 and Presenilin 2 in the neurons of the transgenic zebrafish. Therefore, the present invention provides a transgenic zebrafish where any combination of Tau, APP, amyloid β, apoE, Presenilin 1, and Presenilin 2 is expressed in the neurons of transgenic zebrafish. Transgenic zebrafish that express any combination of mutant Tau, APP, amyloid β, apoE, Presenilin 1 and Presenilin 2, including fragments thereof, are also provided by this invention. For example, the present invention provides a transgenic zebrafish that expresses Tau and APP in the neurons of the transgenic zebrafish. Also provided is a transgenic zebrafish that expresses Tau and amyloid β in the neurons of the transgenic zebrafish. Further provided is a transgenic zebrafish that expresses APP and presenilin 1 in the neurons of the transgenic zebrafish. These examples are merely exemplary and should not be considered limiting as there are numerous combinations of proteins associated with Alzheimer's disease that can be expressed in the transgenic zebrafish of this invention.

Transgenic zebrafish in which the expression of one or more proteins associated with Alzheimer's disease selected from the group consisting of: Tau, Amyloid precursor protein (APP), Amyloid β, Apolipoprotein E (apoE), Presenilin 1 and Presenilin 2 is tissue-specific is contemplated for this invention (see U.S. Pat. No. 6,380,458 which is incorporated herein in its entirety by this reference for the purposes of describing tissue specific expression of a protein in zebrafish). Transgenic zebrafish with tissue specific expression of a reporter protein is also contemplated (see U.S. Pat. No. 6,380,458 which is incorporated herein in its entirety by this reference for the purposes of describing tissue specific expression of a reporter protein in zebrafish). For example, transgenic animals that express a reporter protein, or any other protein associated with Alzheimer's disease at specific sites such as neurons can be produced by introducing a nucleic acid encoding the protein into fertilized eggs, embryonic stem cells or the germline of the animal, wherein the nucleic acid is under the control of a specific promoter which allows expression of the nucleic acid in specific types of cells (e.g., a promoter which allows expression primarily in neurons). As used herein, a protein or gene is expressed predominantly in a given tissue, cell type, cell lineage or cell, when 90% or greater of the observed expression occurs in the given tissue cell type, cell lineage or cell.

More specifically, this invention contemplates the use of a transgenic zebrafish that express a protein that is under the control of the zebrafish GATA-2 promoter and is expressed in neurons. Examples of a zebrafish GATA-2 promoter include, but are not limited to a nucleic acid comprising SEQ ID NO: 10 and a nucleic acid comprising SEQ ID NO: 11. The present invention also provides a transgenic zebrafish that expresses a protein that is under the control of the zebrafish tyrosine hydroxylase promoter and is expressed in catecholaminergic and dopaminergic neurons. The promoters for the tyrosine hydroxylase or dopamine transporter gene (Holzschuh et al., 2001) can also be used to drive dopaminergic neuron-specific expression of a protein. For tissue-specific expression in all or most neurons, expression sequences for the HuC/elav (Park et al., 2000), Thy-1.2, dystrophin, prion, platelet-derived growth factor B-chain, tau, alpha tubulin (Goldman et al., 2001), or beta tubulin (Oehlmann et al., 2004) gene can be utilized. The islet-1 promoter (Higashijima et al., 2000) can be utilized to express a protein in cranial motor neurons of zebrafish. The expression sequences used to drive expression of the proteins described herein can be isolated by one of skill in the art, for example, by screening a genomic zebrafish library for sequences upstream of the zebrafish gene of interest. The expression sequences can include a promoter, an enhancer, a silencer and necessary information processing sites, such as ribosome binding sites, RNA splice sites, polyadenylation sites and transcriptional terminator sequences. For example, the expression sequences can comprise neuronal promoter sequences. The expression sequences can also comprise neuronal enhancer sequences.

The expression sequences of the present invention can also include inducible promoters, such as the inducible promoters of the GAL4/VP16-UAS system (Köster and Fraser, 2001). For example, a construct comprising a neuron specific expression sequence operably linked to a nucleic acid sequence encoding a GAL4/VP16 transcriptional activator and a construct comprising a UAS expression sequence operably linked to a protein associated with Alzheimer's disease can be introduced into a zebrafish embryo to produce a zebrafish that expresses a protein associated with Alzheimer's in the neurons of the transgenic fish upon transcriptional activation by GAL4/VP16. In other words, protein expression is dependent on transcriptional activation by GAL4/VP 16 which is specifically expressed in neurons. Alternatively, the UAS expression sequence operably linked to a protein associated with Alzheimer's disease and the neuron specific expression sequence operably linked to a nucleic acid encoding a GAL4/VP16 transcriptional activator can be introduced into a zebrafish embryo on the same construct. Also, a transgenic zebrafish line comprising a neuron specific promoter driving expression of Gal4/VP16 can be crossed with a second zebrafish line comprising a UAS expression sequence driving expression of a protein associated with Alzheimer's disease in order to obtain progeny containing both constructs. Therefore, these zebrafish can be made using any of the proteins described herein, such as Tau, APP, amyloid β, apoE, Presenilin 1, Presenilin 2 and fragments thereof. These zebrafish can also be made using mutant versions of Tau, APP amyloid β, apoE, Presenilin 1, Presenilin 2 and fragments thereof. Fusion polypeptides comprising Tau, APP, amyloid β, apoE, Presenilin 1, Presenilin 2, and fragments thereof can also be utilized.

Other inducible systems could also be used such as tetracycline inducible constructs or glucocorticoid inducible constructs. A Cre-lox system can also be utilized as an inducible system in the zebrafish of the present invention (See Thummel et al. “Cre-mediated site-specific recombination in zebrafish embryos,” Developmental Dynamics 233: 1366-1377 (2005) and Langenau et al., “Cre/lox-regulated transgenic zebrafish model with conditional myc-induced T cell acute lymphoblastic leukemia,” PNAS 102: 6068-607 (2005), both of which are incorporated in their entireties by this reference.)

The transgenic zebrafish of the present invention can also comprise a nucleic acid encoding a zinc transporter. The nucleic acid encoding a zinc transporter can be on the same construct as the nucleic acid encoding a protein described herein, or it can be on a separate construct. This construct can be introduced simultaneously with the other constructs described herein when making a transgenic fish. Alternatively, a transgenic zebrafish line comprising a nucleic acid encoding a zinc transporter can be crossed with a second zebrafish line comprising a construct that directs neuronal specific expression of a protein associated with Alzheimer's disease in order to obtain progeny containing both constructs. Therefore, these zebrafish can be made using any of the proteins described herein, such as Tau, APP, amyloid β, apoE, Presenilin 1, Presenilin 2 and fragments thereof. These zebrafish can also be made using mutant versions of Tau, APP amyloid f, apoE, Presenilin 1, Presenilin 2 and fragments thereof. Fusion polypeptides comprising Tau, APP, amyloid β, apoE, Presenilin 1, Presenilin 2, and fragments thereof can also be utilized.

As utilized herein, “a pathology associated with Alzheimer's disease” is a characteristic seen in the brain (i.e. histopathology) of Alzheimer's disease sufferers. These characteristics or features do not have to be recapitulated exactly as seen in the brain of a subject with Alzheimer's disease nor does any zebrafish of the present invention have to exhibit all or a specific subset of pathologies associated with Alzheimer's disease. One or more of the characteristics described herein can be observed or detected in the zebrafish of the present invention. These include neuritic plaques and neurofibrillary tangles. Neuritic plaques are insoluble protein deposits that build up around the brain's neurons. Neurofibrillary tangles or aggregates, described as twisted fibers, are also insoluble and are found inside neurons. The plaques are mainly composed of a partial beta-pleated sheet polypeptide, called amyloid beta (βA). The 4.2 kDa polypeptide is cleaved from a large precursor protein, called amyloid precursor protein (APP). Plaques also deposit around neurons of the cerebral cortex, responsible for language and reasoning. In later stages of Alzheimer's disease, neuritic plaques form on many areas of the brain. Therefore, plaque formation in the zebrafish of this invention is not limited to any specific neurons or areas of the brain.

Neurofibrillary tangles, also seen in Alzheimer's disease, contain paired helical filaments composed of the microtubule-associated protein Tau. Therefore, neurofibrillary tangles comprising Tau can be detected in the zebrafish of the present invention as a pathology associated with Alzheimer's disease.

Neuronal damage is also associated with Alzheimer's disease. Alzheimer's disease causes the death of neuronal cells and brain nerves, and disrupts neurotransmitters. For example, a reduction in the number of neurons can occur. This reduction is not limited to specific neurons but can be a reduction in cholinergic neurons, dopaminergic neurons, catecholaminergic neurons hippocampal neurons, forebrain neurons and/or motor neurons. A reduction in the activity of these neurons can also occur. Therefore, damage to neurons, can also be observed or detected as a pathology of Alzheimer's disease in the zebrafish of the present invention.

Other changes in neuronal morphology may also be indicative of Alzheimer's disease pathology. For example, enlarged axonal and dendritic varicosities have been associated with fibrillar Aβ deposits in transgenic mice overexpressing amyloid precurosor protein (Brendza et al., 2003).

Alzheimer's disease is also characterized by memory loss. Assays designed to test memory in fish may also be employed to characterize Alzheimer's disease pathology in zebrafish of the present invention. An example of an assay to test memory in adult and juvenile fish has been described (Williams et al., 2002) and is incorporated herein in its entirety by this reference. Other behavioral or motor assays that indicate neuronal damage may also be contemplated. Examples of behavioral assays in larval zebrafish have been reviewed (see Neuhauss, 2003; Guo, 2004; Saint-Amant and Drapeau, 1998, all of which are incorporated herein in their entireties by this reference).

The transgenic fish utilized in the methods of this invention are produced by introducing a transgenic construct into cells of a zebrafish, preferably embryonic cells, and most preferably in a single cell embryo, essentially as described in Meng et al. (1998). The transgenic construct is preferably integrated into the genome of the zebrafish, however, the construct can also be constructed as an artificial chromosome. The transgenic construct can be introduced into embryonic cells using any technique known in the art or later developed for the introduction of transgenic constructs into embryonic cells. For example, microinjection, electroporation, liposomal delivery and particle gun bombardment can all be utilized to effect transgenic construct delivery to embryonic cells as well as other methods standard in the art for delivery of nucleic acids to zebrafish embryos or embryonic cells. Embryos can be obtained by mating adult zebrafish in specially designed mating tanks. Eggs are usually laid in the morning and are collected immediately so that they can be microinjected at the one cell stage. Embryonic cells can be obtained from zebrafish as described by Fan et al. (2004). Zebrafish containing a transgene can be identified by numerous methods such as probing the genome of the zebrafish for the presence of the transgene construct by Northern or Southern blotting. Polymerase chain reaction techniques can also be employed to detect the presence of the transgene. Expression of Tau, Amyloid precursor protein (APP), amyloid β, Apolipoprotein E (apoE), Presenilin 1 and/or Presenilin 2 can be also be detected by methods known in the art. For example, RNA can be detected using any of numerous nucleic acid detection techniques, such as reverse transcriptase PCR. Alternatively, an antibody can be used to detect the expression of Tau, Amyloid precursor protein (APP), amyloid β, Apolipoprotein E (apoE), Presenilin 1 and/or Presenilin 2. Immunohistochemical stains such as Congo Red (See Sytren et al. (2000) and thioflavin S (see Sun et al. (2002) can also be used to detect protein aggregates such as plaques. One of skill in the art can also utilize other immunohistochemical techniques available in the art and described in the Examples to detect expression of the proteins described herein.

The present invention also provides a transgenic zebrafish that expresses a fusion polypeptide comprising a zebrafish expression sequence operably linked to a sequence encoding a reporter polypeptide and polypeptide selected from the group consisting of Tau, APP, amyloid β, apoE, Presenilin 1 and Presenilin 2, wherein the fusion polypeptide is expressed in the neurons of the transgenic zebrafish and wherein the transgenic zebrafish exhibits a pathology associated with Alzheimer's disease. For example, the present invention provides a transgenic zebrafish that expresses a fusion polypeptide comprising Tau and a reporter polypeptide in the neurons of the transgenic zebrafish. The present invention also provides a transgenic zebrafish that expresses a fusion polypeptide comprising APP and a reporter polypeptide in the neurons of the transgenic zebrafish. Transgenic zebrafish that express more than one fusion polypeptide are also provided. For example, a transgenic zebrafish that expresses 1) a fusion polypeptide comprising Tau and a reporter polypeptide and 2) a fusion polypeptide comprising amyloid β and a reporter polypeptide in the neurons of the transgenic zebrafish is provided herein. Also provided is a transgenic zebrafish that expresses 1) a fusion polypeptide comprising Tau and a reporter polypeptide and 2) a fusion polypeptide comprising APP and a reporter polypeptide in the neurons of the transgenic zebrafish. The reporter polypeptides can be the same or the reporter polypeptides can be different in order to distinguish expression of one polypeptide from another. For example, Tau can be fused to GFP and APP can be fused to red fluorescent polypeptide. As another example, Tau can be fused to red fluorescent polypeptide and APP can be fused to yellow fluorescent polypeptide. These examples are not meant to be limiting as the present invention provides numerous combinations of fusion polypeptides and reporter polypeptides that can be utilized to generate the transgenic zebrafish of the invention.

Transgenic zebrafish that express one or more proteins selected from the group consisting of Tau, APP, amyloid β, apoE, Presenilin 1, Presenilin 2, a Tau protein fragment, an APP protein fragment, an apoE protein fragment, a Presenilin 1 protein fragment or a Presenilin 2 protein fragment, a mutant Tau protein, a mutant APP protein, a mutant amyloid β protein, a mutant apoE protein, a mutant Presenilin 1 protein, and a mutant Presenilin 2 protein in the neurons of the transgenic zebrafish and also express one or more fusion polypeptides comprising a reporter protein and a protein selected from the group consisting of: Tau, APP, amyloid β, apoE, Presenilin 1, Presenilin 2, a Tau protein fragment, an APP protein fragment, an apoE protein fragment, a Presenilin 1 protein fragment or a Presenilin 2 protein fragment, a mutant Tau protein, a mutant APP protein, a mutant amyloid β protein, a mutant apoE protein, a mutant Presenilin 1 protein, or a mutant Presenilin 2 protein in the neurons of the transgenic zebrafish are also provided. Therefore, the zebrafish of the present invention can express one or more of Tau, APP, amyloid β, apoE, Presenilin 1, Presenilin 2, a Tau protein fragment, an APP protein fragment, an apoE protein fragment, a Presenilin 1 protein fragment or a Presenilin 2 protein fragment, a mutant Tau protein, a mutant APP protein, a mutant amyloid, protein, a mutant apoE protein, a mutant Presenilin 1 protein, or a mutant Presenilin 2 protein as well as one or more of Tau, APP, amyloid β, apoE, Presenilin 1, Presenilin 2, a Tau protein fragment, an APP protein fragment, an apoE protein fragment, a Presenilin 1 protein fragment or a Presenilin 2 protein fragment, a mutant Tau protein, a mutant APP protein, a mutant amyloid f protein, a mutant apoE protein, a mutant Presenilin 1 protein, or a mutant Presenilin 2 protein fused to a reporter protein in neurons. These examples are merely exemplary and should not be considered limiting as there are numerous combinations of proteins associated with AD that can be expressed in the transgenic zebrafish of this invention.

As used herein, a reporter protein or reporter polypeptide is any protein that can be specifically detected when expressed. Reporter proteins are useful for detecting or quantitating expression from expression sequences. For example, operatively linking nucleotide sequences encoding a reporter protein to a tissue specific expression sequence allows one to study lineage development, such as the development of neurons. In such studies, the reporter protein serves as a marker for monitoring developmental processes, such as neuronal development, regeneration, neurogenesis and neuronal cell death. The reporter protein can also be used to study neuritic plaques and/or neurofibrillary tangles. Many reporter proteins are known to one of skill in the art. These include, but are not limited to, beta-galactosidase, luciferase, and alkaline phosphatase that produce specific detectable products. Fluorescent reporter proteins can also be used, such as green fluorescent protein (GFP), cyan fluorescent protein (CFP), red fluorescent protein (RFP), yellow fluorescent protein (YFP). Other examples include the green fluorescent protein from Aequorea coerelescens (AcGFP), DsRedExpress, and red coral fluorescent proteins (for example, AmCyan, ZsGreen, ZsYellow, AsRed2, DsRed2, and HcRed1). For example, by utilizing GFP, fluorescence is observed upon exposure to light at 489 nm without the addition of a substrate. The use of a reporter protein that, like GFP, is directly detectable without requiring the addition of exogenous factors are preferred for detecting or assessing gene expression during zebrafish embryonic development. Fluorescent proteins can be isolated from many different species, including but not limited to, Aequorea victoria (Chalfie, et al., 1994), Zoanthus species (Matz, et al., 1999), Renilla reniformis (Ward and Cormier, 1979) and Aequorea coerelescens. The present invention also contemplates utilizing fluorescent reporters that have a short half life in order to monitor damage to the fluorescent neurons of the transgenic zebrafish.

For example, the present invention provides a transgenic zebrafish comprising a nucleic acid construct, the construct comprising a neuron specific expression sequence operably linked to a nucleic acid sequence encoding a fusion polypeptide comprising a Tau polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

Also provided by the present invention is a transgenic zebrafish comprising a nucleic acid construct, the construct comprising a neuron specific expression sequence operably linked to a nucleic acid sequence encoding a fusion polypeptide comprising a Tau polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the Tau polypeptide, wherein the fusion polypeptide is expressed in the neurons of the transgenic zebrafish, and wherein the transgenic zebrafish exhibits a pathology associated with Alzheimer's disease.

This zebrafish allows visualization of neurons via a fluorescent reporter polypeptide and visualization of Tau expression via a second, different fluorescent reporter. For example, neuron specific expression of red fluorescent protein can be utilized with neuron specific expression of a green fluorescent protein/Tau fusion polypeptide to distinguish neurons from the Tau fusion polypeptide. This also allows visual differentiation of neurons and neurofibrillary tangles. In another scenario, neuron specific expression of green fluorescent protein or red fluorescent protein can be utilized to assess neurons in the presence of neuron specific expression of a Tau, APP, amyloid β, apoE, Presenilin 1 or Presenilin 2 protein that is not linked to a fluorescent protein.

As used herein, the term “nucleic acid” refers to single or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the moieties discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides), a reduction in the AT content of AT rich regions, or replacement of non-preferred codon usage of the expression system to preferred codon usage of the expression system. The nucleic acid can be directly cloned into an appropriate vector, or if desired, can be modified to facilitate the subsequent cloning steps. Such modification steps are routine, an example of which is the addition of oligonucleotide linkers which contain restriction sites to the termini of the nucleic acid. General methods are set forth in in Sambrook et al. (2001) Molecular Cloning—A Laboratory Manual (3rd ed.) Vol. 1-3, Cold Spring Harbor Laboratory, Cold Spring Harbor Press, NY, (Sambrook).

Once the nucleic acid sequence is obtained, the sequence encoding the specific amino acids can be modified or changed at any particular amino acid position by techniques well known in the art. For example, PCR primers can be designed which span the amino acid position or positions and which can substitute any amino acid for another amino acid. Alternatively, one skilled in the art can introduce specific mutations at any point in a particular nucleic acid sequence through techniques for point mutagenesis. General methods are set forth in Smith, M. “In vitro mutagenesis” Ann. Rev. Gen., 19:423-462 (1985) and Zoller, M. J. “New molecular biology methods for protein engineering” Curr. Opin. Struct. Biol., 1:605-610 (1991), which are incorporated herein in their entirety for the methods. These techniques can be used to alter the coding sequence without altering the amino acid sequence that is encoded.

Unless otherwise specified, any reference to a nucleic acid molecule includes the reverse complement of the nucleic acid. Any nucleic acid written to depict only a single strand encompasses both strands of a corresponding double-stranded nucleic acid. Additionally, reference to the nucleic acid molecule that encodes a specific protein, or a fragment thereof, encompasses both the sense strand and its reverse complement. The present invention also provides a vector comprising any of the nucleic acids set forth herein. These include vectors for expression in both eukaryotic and prokaryotic host cells, either in vitro, in vivo or ex vivo.

Further provided by the present invention is a transgenic zebrafish comprising a nucleic acid construct, the construct comprising a neuron specific expression sequence operably linked to a nucleic acid sequence encoding a Tau polypeptide wherein the Tau polypeptide is expressed in the neurons of the transgenic zebrafish, and wherein the transgenic zebrafish exhibits a pathology associated with Alzheimer's disease.

As utilized herein, when referring to a Tau protein or polypeptide utilized in the present invention, the Tau protein or polypeptide can be any wildtype or mutant Tau protein from any vertebrate species, including, but not limited to fish (zebrafish, tilapia, goldfish, salmon, fugu, medaka, other teleosts), human or other primate species (chimpanzee, gorilla, orangutan, macaque, gibbon), mouse, dog, cat, rat, frog, pig, hamster, guinea pig, and rabbit. Fragments of Tau proteins and fragments of mutant Tau proteins can also be utilized. Fusion polypeptides comprising a Tau polypeptide, a fragment of a Tau polypeptide, a mutant Tau polypeptide or a fragment of a mutant Tau polypeptide are also provided. Nucleotide sequences encoding any of the Tau proteins or Tau protein fragments described herein are also provided by the present invention. For example, the Tau protein of the present invention can be the human wildtype microtubule associated Tau found under GenBank Accession Nos. NM_{—}005910, NM_{—}016834, NM_{—}016841, AH005895, AF047863, or AY730549. The polypeptide sequences, nucleic acid sequences encoding a Tau polypeptide and the information set forth under GenBank Accession Nos. NM_{—}005910, NM_{—}016834, NM_{—}016841, AH005895, AF047863, and AY730549 are hereby incorporated by reference. Any isoform of Tau may be used for the present invention (described in Buée et al., 2000). Other Tau proteins include, but are not limited to, a Tau protein with one or more mutations selected from the group consisting of: K257T, 1260V, G272V, N279K, delK280, P301L, P301S, S305N, V337M, G389R, R406W. The numbering set forth for these mutations corresponds to the numbering of the wildtype amino acid sequence set forth under NM_{—}005910 (SEQ ID NO: 1). The nucleic acid sequence encoding the sequence set forth under NM_{—}005910 is also set forth herein as SEQ ID NO: 12). The Tau proteins of the present invention can also be the three repeat form of the Tau protein and mutants of the three repeat form of the Tau protein

The amino acid sequence of the three repeat form is as follows:

(SEQ ID NO: 2)

maeprqefevmedhagtyglgdrkdqggytmhqdqegdtdaglkesplqt
ptedgseepgsetsdakstptaedvtaplvdegapgkqaaaqphteipeg
ttaeeagigdtpsledeaaghvtqarmvskskdgtgsddkkakgadgktk
iatprgaappgqkgqanatripaktppapktppssgeppksgdrsgyssp
gspgtpgsrsrtpslptpptrepkkvavvrtppkspssaksrlqtapvpm
pdlknvkskigstenlkhqpgggkvqivykpvdlskvtskcgslgnihhk
pgggqvevksekldfkdrvqskigsldnithvpgggnkkiethkltfren
akaktdhgaeivykspvvsgdtsprhlsnvsstgsidmvdspqlatlade
vsaslakgql

For example, the Tau protein of the present invention can be the three repeat form of human Tau (SEQ ID NO: 2) comprising one or more mutations selected from the group consisting of K257T, 1260V, G272V. Therefore, the present invention also provides constructs comprising a nucleotide sequence encoding SEQ ID NO: 2 or mutant versions of SEQ ID NO: 2. The protein of the present invention can also be a zebrafish Tau protein. For example, the zebrafish Tau protein of the present invention can be the zebrafish Tau protein found under GenBank Accession No.

BI981282, BI1878304, BF937789 or CK400786. These sequences and the information contained under GenBank Accession Nos. BI981282, BI1878304, BF937789 and CK400786 are incorporated herein by this reference. These sequences are zebrafish Tau protein fragments that are between 56%-75% identical to human Tau at the amino acid level.

Also provided by the present invention is a transgenic zebrafish comprising a nucleic acid construct, the construct comprising a neuron specific expression sequence operably linked to a nucleic acid sequence encoding a fusion polypeptide comprising an APP polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

Also provided by the present invention is a transgenic zebrafish comprising a nucleic acid construct, the construct comprising a neuron specific expression sequence operably linked to a nucleic acid sequence encoding a fusion polypeptide comprising a APP polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the APP polypeptide, wherein the fusion polypeptide is expressed in the neurons of the transgenic zebrafish, and wherein the transgenic zebrafish exhibits a pathology associated with Alzheimer's disease.

This zebrafish allows visualization of neurons via a fluorescent reporter polypeptide and visualization of APP expression via a second, different fluorescent reporter. For example, neuron specific expression of green fluorescent protein can be utilized with neuron specific expression of a red fluorescent protein/APP fusion polypeptide to distinguish neurons from the APP fusion polypeptide. This also allows visual differentiation of neurons and neuritic plaques. Furthermore, co-localization of fluorescent neurons with fluorescent fusion polypeptides allows visualization of changes in neurons that result from overexpression of Alzheimer's disease proteins.

Further provided by the present invention is a transgenic zebrafish comprising a nucleic acid construct, the construct comprising a neuron specific expression sequence operably linked to a nucleic acid sequence encoding an APP polypeptide wherein the APP polypeptide is expressed in the neurons of the transgenic zebrafish, and wherein the transgenic zebrafish exhibits a pathology associated with Alzheimer's disease.

As utilized herein, when referring to an APP protein or polypeptide of the present invention, the APP protein or APP polypeptide can be any wildtype isoform or mutant APP protein from any vertebrate species, including, but not limited to human or other primate species, fish (zebrafish, tilapia, goldfish, salmon), mouse, dog, cat, rat, frog pig, hamster, guinea pig, and rabbit. Fragments of APP proteins are also contemplated. Fragments of APP proteins and mutant fragments of APP proteins are also contemplated. Fusion polypeptides comprising an APP polypeptide, a fragment of an APP polypeptide, a mutant APP polypeptide or a fragment of a mutant APP polypeptide are also provided. Nucleic acid sequences encoding any of the APP polypeptides or fragments set forth herein are also provided. For example, the APP protein of the present invention, can be the human wildtype APP (isofomi c) found under GenBank Accession No. NM_{—}201414 (SEQ ID NO: 3). The nucleic acid sequence encoding APP can also be found under GenBank Accession No. NM_{—}201414 and is set forth herein as SEQ ID NO: 13. The polypeptide sequences, nucleic acid sequences and the information set forth under GenBank Accession No. NM_{—}201414 are hereby incorporated by reference. Other variants of APP may also be used including those found under the following GenBank Accession Nos: NM_{—}201413, NM_{—}000484, and AH005295. Other APP proteins include, but are not limited to a human APP protein with one or more mutations selected from the group consisting of: Glu665D, K 670N/M671L, A673T, H677R, D678N, A692G, Glu693G, Glu693Q, D694N, A713T, A713V, T7141, T715A, V715M, V715A, 1716V, 1716T, V717F, V717G, V7171, V717L, and L723P. The numbering set forth for these mutations corresponds to the numbering of the wildtype amino acid sequence set forth under GenBank Accession No. AH005295. GenBank Accession No. AH005295 corresponds to the full length APP (SEQ ID NO: 4). This sequence and the information set forth under GenBank Accession No. AH005295 are hereby incorporated by reference. The nucleic acid sequence encoding the full length APP is also set forth herein as SEQ ID NO: 14.

Also provided by the present invention is a transgenic zebrafish comprising a nucleic acid construct, the construct comprising a neuron specific expression sequence operably linked to a nucleic acid sequence encoding a fusion polypeptide comprising a presenilin polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

Also provided by the present invention is a transgenic zebrafish comprising a nucleic acid construct, the construct comprising a neuron specific expression sequence operably linked to a nucleic acid sequence encoding a fusion polypeptide comprising a presenilin polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the presenilin polypeptide, wherein the fusion polypeptide is expressed in the neurons of the transgenic zebrafish, and wherein the transgenic zebrafish exhibits a pathology associated with Alzheimer's disease.

Such a zebrafish allows visualization of neurons via a fluorescent reporter polypeptide and visualization of presenilin expression via a second, different fluorescent reporter. For example, neuron specific expression of green fluorescent protein can be utilized with neuron specific expression of a red fluorescent protein/presenilin fusion polypeptide to distinguish neurons from the presenilin fusion polypeptide.

Further provided by the present invention is a transgenic zebrafish comprising a nucleic acid construct, the construct comprising a neuron specific expression sequence operably linked to a nucleic acid sequence encoding a presenilin polypeptide wherein the presenilin polypeptide is expressed in the neurons of the transgenic zebrafish, and wherein the transgenic zebrafish exhibits a pathology associated with Alzheimer's disease.

The presenilin proteins of the present invention include presenilin 1 and presenilin 2 proteins. As utilized herein, when referring to a presenilin protein or polypeptide of the present invention, the presenilin protein or polypeptide can be any wildtype or mutant presenilin protein from any vertebrate species, including, but not limited to human or other primate species, fish (zebrafish, tilapia, goldfish, salmon), mouse, dog, cat, rat, frog pig, hamster, guinea pig, and rabbit. Fragments of presenilin proteins and fragments of mutant presenilin proteins are also contemplated. Fusion polypeptides comprising a presenilin polypeptide, a fragment of a presenilin polypeptide, a mutant presenilin polypeptide or a fragment of a mutant presenilin polypeptide are also provided. Nucleic acid sequences encoding the presenilin polypeptides of the present invention are also provided herein.

For example, the presenilin 1 protein of the present invention can be the human wildtype presenilin 1 found under GenBank Accession No. NM_{—}000021 (SEQ ID NO: 5)

The nucleic acid sequence encoding presenilin 1 (SEQ ID NO: 15) can also be found under GenBank Accession No. NM_{—}000021. The polypeptide sequences, nucleic acid sequences and the information set forth under GenBank Accession No. NM_{—}000021 are hereby incorporated by reference. Other presenilin 1 proteins include, but are not limited to a human presenilin 1 protein with one or more mutations selected from the group consisting of: A79V, V82L, L85P, C92S, V94M, V96F, F105L, Y115C, Y115H, T116N, P117L, P117R, E120D, E120D2, E120K, E123K, N135D, M1391, M139T, M139V, 1143F, 1143M, 1143T, M146I, M146L, M146V, T147L, H163R, H163Y, W165C, S169L, S169P, L171P, L173W, L174M, G183V, E184D, G209V, 1213F, 1213T, L219F, L219P, Q222H, L226R, A231T, A231V, M233L, M233T, L235P, F2371, A246E, L250S, Y256S, A260V, V261F, L262F, C263R, P264L, P267S, R269G, R269H, E273A, R278T, E280A, E280G, L282R, A285V, L286V, S290C, S290C2, S290C3, G378E, G384A, S3901, L392V, N405S, A409T, C410Y, L424R, A426P, P436Q and P436S. The numbering set forth for these mutations corresponds to the numbering of the wildtype amino acid sequence set forth under NM_{—}000021.

The presenilin 2 protein of the present invention can be the human wildtype presenilin 2 found under GenBank Accession No. NM_{—}000447 (SEQ ID NO: 6). The polypeptide sequences, nucleic acid sequences and the information set forth under GenBank Accession No. NM_{—}000447 are hereby incorporated by reference. The nucleic acid sequence encoding presenilin 2 is also set forth herein as SEQ ID NO: 16. Other presenilin 2 proteins include, but are not limited to a human presenilin 2 protein with one or more mutations selected from the group consisting of: R62H, T122P, S130L, N1411, V1481, Q228L, M2391 and M239V. The numbering set forth for these mutations corresponds to the numbering of the wildtype amino acid sequence set forth under NM_{—}000447 (SEQ ID NO:6).

Also provided by the present invention is a transgenic zebrafish comprising a nucleic acid construct, the construct comprising a neuron specific expression sequence operably linked to a nucleic acid sequence encoding a fusion polypeptide comprising a amyloid β polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

Also provided by the present invention is a transgenic zebrafish comprising a nucleic acid construct, the construct comprising a neuron specific expression sequence operably linked to a nucleic acid sequence encoding a fusion polypeptide comprising a amyloid β polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the amyloid β polypeptide, wherein the fusion polypeptide is expressed in the neurons of the transgenic zebrafish, and wherein the transgenic zebrafish exhibits a pathology associated with Alzheimer's disease.

This zebrafish allows visualization of neurons via a fluorescent reporter polypeptide and visualization of presenilin expression via a second, different fluorescent reporter. For example, neuron specific expression of green fluorescent protein can be utilized with neuron specific expression of a red fluorescent protein/amyloid β fusion polypeptide to distinguish neurons from the amyloid β fusion polypeptide.

Further provided by the present invention is a transgenic zebrafish comprising a nucleic acid construct, the construct comprising a neuron specific expression sequence operably linked to a nucleic acid sequence encoding an amyloid β polypeptide wherein the amyloid β polypeptide is expressed in the neurons of the transgenic zebrafish, and wherein the transgenic zebrafish exhibits a pathology associated with Alzheimer's disease.

As utilized herein, when referring to an amyloid β protein or polypeptide of the present invention, the amyloid β protein or polypeptide can be any wildtype or mutant amyloid β protein from any vertebrate species, including, but not limited to human or other human primates, fish (zebrafish, tilapia, goldfish, salmon), mouse, dog, cat, rat, frog pig, hamster, guinea pig, and rabbit. Fragments of amyloid β proteins are also contemplated. Fusion polypeptides comprising an amyloid β polypeptide, a fragment of an amyloid β polypeptide, a mutant amyloid β polypeptide or a fragment of a mutant amyloid β polypeptide are also provided. Nucleic acids encoding the amyloid β proteins or polypeptides set forth herein are also provided. For example, the amyloid β protein of the present invention can be the human wildtype amyloid β42 peptide with the following sequence of 42 amino acids:

(SEQ ID NO: 7)

DAEFRHDSGYEVHHQKLVFFAEDVGSNKGAIIGLMVGGVVIA.

Other amyloid β proteins include, but are not limited to a human amyloid β protein with one or more mutations selected from the group consisting of: Aβ42 peptide, Arctic mutant (E22G), Aβ42 peptide, Flemish mutant (A21 G), Aβ42 peptide, Dutch mutant (E22Q), Aβ42 peptide, Italian mutant (E22K), Aβ42 peptide and Iowa mutant (D23N). The numbering set forth for these mutations corresponds to the numbering of the wildtype amino acid sequence set forth above.

As stated above, the present invention also provides nontransgenic zebrafish that can be manipulated to express or overexpress a polypeptide associated with AD, by directly administering a polypeptide associated with AD or a fragment thereof to a zebrafish. For example, the present invention also provides zebrafish in which the amyloid β polypeptides are introduced into the brain of the zebrafish, for example, by intracerebroventricular infusion (See Craft et al. “Aminopyridazines inhibit beta-amyloid-induced glial activation and neuronal damage in vivo” Neurobiology of Aging 25: 1283-1292 (2004) which is incorporated herein in its entirety by this reference.). These nontransgenic zebrafish can be utilized in the methods described herein to identify compounds that modulate a pathology of Alzheimer's disease.

### Screening Methods

Any of the transgenic zebrafish described herein that express one or more proteins selected from the group consisting of Tau, APP, amyloid β, apoE, Presenilin 1 and Presenilin 2 in the neurons of the zebrafish can be utilized to screen for agents that modulate a pathology associated with Alzheimer's disease. These include transgenic zebrafish that express one or more fusion polypeptides comprising a reporter polypeptide and a protein selected from the group consisting of Tau, APP, amyloid β, apoE, Presenilin 1 and Presenilin 2.

By “modulate” is meant any change in a pathology associated with Alzheimer's disease. As discussed above, these include but are not limited to a change in neuronal activity, a change in the number of neurons, a change in neuronal damage, a change in neuritic plaques, a change in neurofibrillary tangles, a change in neuronal morphology, a changes in behavior, a changes in memory and the like. A change can be an increase or a decrease and does not have to be complete. For example, there can be a change of 0.01%, 0.1%, 1%, 2%, 5%, 10%, 15%, 20%, 25%, 30%, 35%, 40%, 45%, 50%, 55%, 60%, 65%, 70%, 75%, 80%, 85%, 90%, 95%, 87%, 99%, 100% or any percentage in between. If modulation involves an increase, this increase can be greater than 100%. As discussed above, since pathologies associated with AD can be visualized, one of skill in the art can also assess whether or not a change has occurred via qualitative means.

For example, the present invention provides a method of identifying an agent that modulates a pathology associated with Alzheimer's disease comprising: a) contacting a transgenic zebrafish that expresses a Tau polypeptide comprising a zebrafish neuron specific expression sequence operably linked to a sequence encoding a Tau polypeptide, wherein the Tau polypeptide is expressed in the neurons of the transgenic zebrafish and wherein the transgenic zebrafish exhibits a pathology associated with Alzheimer's disease with a test agent; b) comparing the neuronal pathology of the zebrafish contacted with the test agent to the neuronal pathology of a transgenic zebrafish that expresses Tau polypeptide in its neurons and was not contacted with the test agent; and c) determining the effect of the test agent on the zebrafish, such that if there is a difference in the neuronal pathology of the zebrafish contacted with the test agent and the zebrafish not contacted with the test agent, the test agent is an agent that modulates a pathology associated with Alzheimer's disease.

The method described above can also be performed with a transgenic zebrafish comprising a nucleic acid construct, the construct comprising a neuron specific expression sequence operably linked to a nucleic acid sequence encoding a fusion polypeptide comprising a Tau polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

The method described above can also be performed with a transgenic zebrafish comprising a nucleic acid construct, the construct comprising a neuron specific expression sequence operably linked to a nucleic acid sequence encoding a reporter polypeptide, wherein the reporter polypeptide is expressed in the neurons of the transgenic zebrafish. Compound screening in this transgenic fish can identify compounds that affect the proliferation or survival of neurons in the absence of an Alzheimer's disease pathology. This method can also be performed with a transgenic zebrafish comprising a nucleic acid construct, the construct comprising a neuron specific expression sequence operably linked to a nucleic acid sequence encoding a fusion polypeptide comprising a Tau polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the Tau polypeptide, wherein the fusion polypeptide is expressed in the neurons of the transgenic zebrafish, and wherein the transgenic zebrafish exhibits a pathology associated with Alzheimer's disease.

The test compounds used in the methods described herein can be, but are not limited to, chemicals, small molecules, inorganic molecules, organic molecules, drugs, proteins, cDNAs encoding proteins, secreted proteins, large molecules, antibodies, morpholinos, triple helix molecule, a peptide, siRNAs, shRNAs, miRNAs, antisense RNAs, ribozymes. The zebrafish can be soaked in the test compound or injected with the test compound. Test compounds can be injected into the yolk, introduced into the blood stream by injecting into the heart cavity, injected into the gut or injected intramuscularly. Test compounds comprising nucleic acids can be delivered as naked nucleic acids, or in a vector via methods known in the art. Libraries of compounds can be tested by arraying zebrafish in multi-well plates and administering compounds in small volumes to each well.

In the methods of the present invention, one or more pathologies associated with Alzheimer's disease can be assessed. The effects of the test compound can be assessed, for example, by observing detectable changes in fluorescence, in situ hybridization signal, or immunohistochemical signal. For example, one of skill in the art can compare Tau expression in the transgenic zebrafish contacted with the test compound with Tau expression in the transgenic zebrafish not contacted with the text compound. In the methods of the present invention, expression can be measured by in situ hybridization, via immunohistochemical signal or via other methods such as PCR. A variety of PCR techniques are familiar to those skilled in the art. For a review of PCR technology, see the publication entitled “PCR Methods and Applications” (1991, Cold Spring Harbor Laboratory Press), which is incorporated herein by reference in its entirety for amplification methods. Real-time PCR can also be utilized. In each of these PCR procedures, PCR primers on either side of the nucleic acid sequences to be amplified are added to a suitably prepared nucleic acid sample along with dNTPs and a thermostable polymerase such as Taq polymerase, Pfu polymerase, or Vent polymerase. The nucleic acid in the sample is denatured and the PCR primers are specifically hybridized to complementary nucleic acid sequences in the sample. The hybridized primers are extended. Thereafter, another cycle of denaturation, hybridization, and extension is initiated. The cycles are repeated multiple times to produce an amplified fragment containing the nucleic acid sequence between the primer sites. PCR has further been described in several patents including U.S. Pat. Nos. 4,683,195, 4,683,202 and 4,965,188. Each of these publications is incorporated herein by reference in its entirety for PCR methods.

A detectable label may be included in an amplification reaction. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine (ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g., ³²P, ³⁵S, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified fragment, can be analyzed by one of a number of methods known in the art. The nucleic acid can be sequenced by dideoxy or other methods. Hybridization with the sequence can also be used to determine its presence, by Southern blots, dot blots, etc.

If the Tau protein is fused to a fluorescent reporter protein, changes in Tau expression and/or conformation can be measured via fluorescence. These changes in expression can be decreases or increases in mRNA, decreases or increases in protein expression or changes in protein conformation, such as tangle morphology. Anti-Tau antibodies can be utilized to assess Tau expression and to detect the presence of neurofibrillary tangles. The changes in Tau expression can also be associated with changes in the quantity and quality of neurofibrillary tangles. For example, if upon contacting the transgenic zebrafish with a test compound, fewer neurofibrillary tangles are observed as compared to a control, via fluorescence or other means described herein, this compound modulates a pathology associated with Alzheimer's disease. Similarly, if upon contacting the transgenic zebrafish with a test compound, the quality of the neurofibrillary tangles changes, either by changing the size of the tangles, disrupting the tangles or changing the consistency of the tangles, this compound modulates a pathology of Alzheimer's disease.

For all of the methods of the present invention, the effect of the test compounds on the neurons and neuronal activity of the transgenic zebrafish can also be assessed. Neuronal damage is associated with Alzheimer's disease and can range from decreased neuronal activity to total ablation of neurons. In order to assess the effect of test compounds on damaged neurons, one skilled in the art could determine how much neuronal damage had occurred in the transgenic zebrafish prior to administration of the test compound by, for example, observing whether or not there is any fluorescent reporter protein production in neurons. Alternatively, one of skill in the art could assess neuronal damage via microscopy, immunohistochemical means or in situ hybridization.

Upon administration of the test compound, if an increase in fluorescence occurs in the previously damaged neurons, neuronal regeneration has occurred. Neuronal regeneration is defined as repair or replacement of damaged neurons. If increased fluorescence is observed in neurons previously observed to be expressing no fluorescent reporter protein or a small amount of a fluorescent protein, the test compound is a neuroregenerative compound. Both axons and cell bodies can be monitored in this way. Neuronal regeneration can also be assessed via microscopy, immunohistochemical means or in situ hybridization.

One of skill in the art can also determine if the test compounds promote neurogenesis. As used herein, neurogenesis is defined as proliferation of neurons. In order to assess neurogenesis, one skilled in the art could determine how much neuronal damage had occurred in the zebrafish by, for example, observing how many, if any neurons are expressing a fluorescent reporter protein. Neurons can also be detected using immunohistochemical techniques or in situ hybridization. Upon administration of the test compound, if there is an increase in the number of neurons expressing the fluorescent protein, neurogenesis has occurred and the test compound promotes neurogenesis. Neurogenesis can also be assessed via microscopy, immunohistochemical means or in situ hybridization.

Behavioral phenotypes, such as memory loss, may also be observed in zebrafish of the present invention. If such a phenotype is altered by a compound, such as by decreasing memory loss, then this compound modulates a pathology of Alzheimer's disease. One of skill in the art can assess the effects of a test compound on one or more pathologies associated with Alzheimer's disease.

The present invention also provides a method of identifying an agent that modulates neuronal pathology comprising: a) administering a test agent to a transgenic zebrafish expressing a reporter protein in neurons, b) comparing the expression of the reporter protein in the neurons of the zebrafish contacted with the test agent with the expression of the reporter protein in the neurons of a transgenic zebrafish that was not contacted with the test agent; and c) determining the effect of the test compound on the expression of the reporter protein in the neurons, such that if the number of neurons in the zebrafish contacted with the test agent is greater than the number of neurons in the zebrafish that was not contacted with the test agent, the test agent is an neuroproliferative agent.

This method can be performed with a transgenic zebrafish comprising a nucleic acid construct, the construct comprising a neuron specific expression sequence operably linked to a reporter protein.

Therefore, a test agent can be administered to a transgenic zebrafish expressing a reporter protein in neurons, wherein the zebrafish does not exhibit a pathology of Alzheimer's Disease. Agents that are found to be neuroproliferative can also be administered to a transgenic zebrafish described herein that exhibits a pathology of Alzheimer's Disease in order to determine if the neuroproliferative agent is also neuroproliferative in a transgenic zebrafish exhibiting a pathology of Alzheimer's Disease.

The effect(s) of a test agent on a transgenic zebrafish expressing a reporter protein in neurons, wherein the zebrafish does not exhibit a pathology of Alzheimer's Disease can also be used as a control for comparing the effect(s) of a test agent on a transgenic zebrafish described herein that exhibits a pathology of Alzheimer's Disease. Similarly, the effects of a test agent on the neurons of a nontransgenic zebrafish that does not exhibit a pathology of Alzheimer's Disease can be used as a control. That is, test agents could affect the proliferation or survival of neurons in a wildtype environment, in the absence of a pathology of Alzheimer's disease. Compounds that are found to promote the growth or survival of neurons in a wildtype environment could have therapeutic potential.

The present invention also provides a method of identifying an agent that modulates a pathology associated with Alzheimer's disease comprising: a) contacting a transgenic zebrafish that expresses an APP polypeptide comprising a zebrafish neuron specific expression sequence operably linked to a sequence encoding an APP polypeptide, wherein the APP polypeptide is expressed in the neurons of the transgenic zebrafish and wherein the transgenic zebrafish exhibits a pathology associated with Alzheimer's disease with a test agent; b) comparing the neuronal pathology of the zebrafish contacted with the test agent to the neuronal pathology of a transgenic zebrafish that expresses an APP polypeptide in its neurons and was not contacted with the test agent; and c) determining the effect of the test agent on the zebrafish, such that if there is a difference in the neuronal pathology of the zebrafish contacted with the test agent and the zebrafish not contacted with the test agent, the test agent is an agent that modulates a pathology associated with Alzheimer's disease.

The method described above can also be performed with a transgenic zebrafish comprising a nucleic acid construct, the construct comprising a neuron specific expression sequence operably linked to a nucleic acid sequence encoding a fusion polypeptide comprising a APP polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

This method can also be performed with a transgenic zebrafish comprising a nucleic acid construct, the construct comprising a neuron specific expression sequence operably linked to a nucleic acid sequence encoding a fusion polypeptide comprising a APP polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the APP polypeptide, wherein the fusion polypeptide is expressed in the neurons of the transgenic zebrafish, and wherein the transgenic zebrafish exhibits a pathology associated with Alzheimer's disease.

As stated above one or more pathologies associated with Alzheimer's disease can be assessed. The effects of the test compound can be assessed by observing detectable changes in fluorescence, in situ hybridization signal, or immunohistochemical signal. For example, one of skill in the art can compare APP expression in the transgenic zebrafish contacted with the test compound with APP expression in the transgenic zebrafish not contacted with the text compound. Expression can be measured by in situ hybridization or via immunohistochemical signal. Expression can also be measured utilizing numerous PCR techniques known in the art. If the APP protein is fused to a fluorescent reporter protein, changes in APP expression can be measured via fluorescence. These changes in expression can be decreases or increases in mRNA or protein expression.

Anti-APP antibodies can be utilized to assess APP expression and to detect the presence of neuritic plaques. Histochemical stains such as Congo Red and thioflavin S may also be used. The changes in APP expression can also be associated with changes in the quantity and quality of neuritic plaques. For example, if upon contacting the transgenic zebrafish with a test compound, fewer neuritic plaques are observed as compared to a control, via fluorescence or other means described herein, this compound modulates a pathology associated with Alzheimer's disease. Similarly, if upon contacting the transgenic zebrafish with a test compound, the quality of the neuritic plaques changes, either by changing the size of the plaques, their morphology or their consistency, this compound modulates a pathology of Alzheimer's disease.

The present invention also provides a method of identifying an agent that modulates a pathology associated with Alzheimer's disease comprising: a) contacting a transgenic zebrafish that expresses an amyloid β polypeptide comprising a zebrafish neuron specific expression sequence operably linked to a sequence encoding an amyloid β polypeptide, wherein the amyloid β polypeptide is expressed in the neurons of the transgenic zebrafish and wherein the transgenic zebrafish exhibits a pathology associated with Alzheimer's disease with a test agent; b) comparing the neuronal pathology of the zebrafish contacted with the test agent to the neurons of a transgenic zebrafish that expresses an APP polypeptide in its neurons and was not contacted with the test agent; and c) determining the effect of the test agent on the zebrafish, such that if there is a difference in the neuronal pathology of the zebrafish contacted with the test agent and the zebrafish not contacted with the test agent, the test agent is an agent that modulates a pathology associated with Alzheimer's disease.

The method described above can also be performed with a transgenic zebrafish comprising a nucleic acid construct, the construct comprising a neuron specific expression sequence operably linked to a nucleic acid sequence encoding a fusion polypeptide comprising an amyloid β polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

This method can also be performed with a transgenic zebrafish comprising a nucleic acid construct, the construct comprising a neuron specific expression sequence operably linked to a nucleic acid sequence encoding a fusion polypeptide comprising a an amyloid β polypeptide and a fluorescent reporter polypeptide, and further comprising a second nucleic acid construct comprising a neuron specific expression sequence operably linked to a nucleic acid sequence encoding a fluorescent reporter polypeptide that is different from the reporter polypeptide fused to the amyloid β polypeptide, wherein the fusion polypeptide is expressed in the neurons of the transgenic zebrafish, and wherein the transgenic zebrafish exhibits a pathology associated with Alzheimer's disease.

The effects of the test compound can be assessed by observing detectable changes in fluorescence, in situ hybridization signal, or immunohistochemical signal. For example, one of skill in the art can compare amyloid β expression in the transgenic zebrafish contacted with the test compound with amyloid β expression in the transgenic zebrafish not contacted with the test compound. Expression can be measured by in situ hybridization or via immunohistochemical signal, or by utilizing PCR techniques known in the art. If the amyloid f protein is fused to a fluorescent reporter protein, changes in amyloid β expression can be measured via fluorescence. These changes in expression can be decreases or increases in mRNA or protein expression.

Anti-amyloid β antibodies can be utilized to assess amyloid β expression and to detect the presence of neuritic plaques. The changes in amyloid β expression can also be associated with changes in the quantity and quality of neuritic plaques. For example, if upon contacting the transgenic zebrafish with a test compound, fewer neuritic plaques are observed as compared to a control, via fluorescence or other means described herein, this compound modulates a pathology associated with Alzheimer's disease. Similarly, if upon contacting the transgenic zebrafish with a test compound, the quality of the neuritic plaques changes, either by changing the size of the plaques, or their consistency, this compound modulates a pathology of Alzheimer's disease.

As mentioned above, the methods of the present invention can be utilized with any of the transgenic zebrafish described herein. Therefore, the present invention also provides methods of identifying agents that modulate a pathology of Alzheimer's disease by utilizing transgenic zebrafish described herein that express apoE, presenilin 1 or presenilin 2 in neurons. The methods of detection described herein can also be utilized with transgenic zebrafish expressing apoE, presenilin 1 or presenilin 2. All of the pathologies associated with Alzheimer's disease can also be assessed using transgenic zebrafish expressing apoE, presenilin 1 or presenilin 2. As discussed above, the invention provides zebrafish wherein more than one protein selected from the group consisting of Tau, APP, amyloid β. apoE, presenilin 1 and presenilin 2 are expressed in the neurons of a transgenic zebrafish. Therefore, the present invention provides screening methods wherein a transgenic zebrafish expressing more than one protein selected from the group consisting of Tau, APP, amyloid β. apoE, presenilin 1 and presenilin 2 is contacted with a test compound and its effects on a pathology associated with Alzheimer's disease is assessed. For example, one of skill in the art can make a transgenic zebrafish expressing Tau and APP in neurons as described herein, contact this zebrafish with a test compound and assess the effects of the compound on a pathology of Alzheimer's disease. In this case, Tau and/or APP expression can be assessed. The effects of the compound on neuritic plaques and/or neurofibrillary tangles can also be assessed. Furthermore, the effects of the compound on neurons and/or neuronal activity can also be assessed as described above. Similarly, one of skill in the art can make a transgenic zebrafish expressing Tau and amyloid f in neurons, contact this zebrafish with a test compound and assess the effects of the compound on a pathology of Alzheimer's disease. These examples are not meant to be limiting as there are numerous combinations of proteins associated with Alzheimer's disease that one of skill in the art can use to make the transgenic zebrafish of this invention and identify compounds that modulate a pathology of Alzheimer's disease.

Those compounds found to modulate a pathology of Alzheimer's disease can be utilized to treat Alzheimer's disease. Furthermore, compounds can be utilized in other in vivo animal models of Alzheimer's disease such as a mouse model, a rat model or a rabbit model to study their therapeutic effects. For example, a compound identified by the methods of the present invention can be utilized in a mouse model to assess its in vivo effects on pathologies associated with Alzheimer's disease.

One of skill in the art will know that the compounds of the present invention can be administered to a subject in a suitably acceptable pharmaceutical carrier. The subject can be any mammal, preferably human, and can include, but is not limited to mouse, rat, cow, guinea pig, hamster, rabbit, cat, dog, goat, sheep, monkey, horse and chimpanzee. By pharmaceutically acceptable is meant a material that is not biologically or otherwise undesirable, i.e., the material may be administered to an individual along with the selected agent without causing any undesirable biological effects or interacting in a deleterious manner with any of the other components of the pharmaceutical composition in which it is contained. In addition, one can include other medicinal agents, pharmaceutical agents, carriers, adjuvants, diluents, etc.

The compounds of the present invention can be administered via oral administration, nebulization, inhalation, mucosal administration, intranasal administration, intratracheal administration, intravenous administration, intraperitoneal administration, subcutaneous administration, intracerebral delivery (such as intracerebral injection or by convection enhanced delivery) and intramuscular administration.

Dosages of the compositions of the present invention will also depend upon the type and/or severity of the disease and the individual subject's status (e.g., species, weight, disease state, etc.) Dosages will also depend upon the form of the composition being administered and the mode of administration. Such dosages are known in the art or can be determined by one of skill in the art.

Furthermore, the dosage can be adjusted according to the typical dosage for the specific disease or condition to be treated. Often a single dose can be sufficient; however, the dose can be repeated if desirable. The dosage should not be so large as to cause adverse side effects. Generally, the dosage will vary with the age, condition, sex and other parameters and can be determined by one of skill in the art according to routine methods (see e.g., Remington's Pharmaceutical Sciences). The individual physician in the event of any complication can also adjust the dosage.

### Target Identification and Validation

Also provided by the present invention is a method of identifying and/or validating genes involved in Alzheimer's disease. Genes to be tested for function in zebrafish Alzheimer's disease models include genes found in zebrafish cDNA libraries, including neuron-specific cDNA libraries, genes found in zebrafish expressed sequence tag (EST) databases, and genes that are identified as homologues of human genes that may be relevant to Alzheimer's disease. Upon identification of zebrafish genes that are potentially involved in Alzheimer's disease, one of skill in the art would know how to compare the zebrafish sequence with other sequences in available databases in order to identify a human homologue of a neuron specific zebrafish gene. One of skill in the art would also be able to identify other homologues such as a mouse homologue or a rat homologue. Alternatively, sequences from the zebrafish gene can be utilized as probes to screen a human library and identify human homologues. The zebrafish sequences can also be utilized to screen other animal libraries, such as a mouse library or a rat library. Upon identification of a mouse, rat or other animal homologue, these sequences can be utilized to screen for a human homologue, either by searching available databases, or screening a human library.

Upon identification of a gene potentially involved in Alzheimer's disease, the present invention also contemplates knocking out, knocking down or overexpressing genes in zebrafish in order to determine their role in Alzheimer's disease. For example, a transgenic zebrafish of the present invention that expresses a protein associated with Alzheimer's disease in neurons can also have a gene of interest knocked out, knocked down or overexpressed. One of skill in the art would compare embryonic development of this fish with a transgenic zebrafish expressing a protein associated with Alzheimer's disease in neurons that does not have the neuron-specific gene knocked out, knocked down or overexpressed. If there is a difference in a pathology associated with Alzheimer's disease, the gene that has been knocked out, knocked down or overexpressed plays a role in Alzheimer's disease. The differences observed can be in neuronal development, neuronal regeneration, neurogenesis, neuronal cell death, expression of a protein involved in Alzheimer's disease, neurofibrillary tangles and/or neuritic plaques.

Genes can be knocked down in the zebrafish by using antisense morpholinos, peptide nucleic acids, or small interfering RNA (siRNA). Antisense molecules can be injected into embryos at the one cell stage and phenotypes detected for several days thereafter. Genes may also be knocked out using any state of the art technology, such as homologous recombination. Genes may be overexpressed by injecting cDNA constructs into embryos at the one cell stage. Transient overexpression or stable overexpression is contemplated.

Also provided by the present invention is a method of identifying a gene as a target for a compound that modulates a pathology associated with Alzheimer's disease comprising: a) contacting a transgenic zebrafish that expresses a protein associated with Alzheimer's disease in neurons and has a gene knocked out or knocked down, with a compound that modulates a pathology of Alzheimer's disease; b) comparing the neurons of the transgenic zebrafish that does not have a gene knocked out or knocked down and has been contacted with the compound, with the neurons of the transgenic zebrafish with a gene knocked out or knocked down; and d) determining the effect of the compound, such that if the neurons of the transgenic zebrafish that does not have a gene knocked out are different from than the neurons in the knockout zebrafish, the gene is a target for a compound that modulates a pathology of Alzheimer's disease.

Genes associated with Alzheimer's disease identified using the methods of this invention may also form the basis of new models of Alzheimer's disease.

The present invention is more particularly described in the following examples which are intended as illustrative only since numerous modifications and variations therein will be apparent to those skilled in the art.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EXAMPLES

The pathology of Alzheimer's disease (AD) includes the presence of protein aggregates that form plaques and tangles in the brain. Amyloid beta (Aβ) is a major component of extracellular plaques and intracellular tangles are mainly composed of Tau. To recapitulate AD pathology in zebrafish, Aβ and Tau isoforms, for example from human, can be expressed in a neuron-specific manner. The present invention provides zebrafish overexpressing Aβ and Tau isoforms that can be utilized to detect protein aggregation.

### DNA Constructs Expressing Human Tau Isoforms in Zebrafish Neurons In Vivo.

Constructs comprise the zebrafish promoter for the neuron-specific gene elav, a Gal4/VP16-UAS construct to enhance transient expression of transgenes, and various isoforms of human Tau fused to a green fluorescent protein derived from Aequorea coerulescens (AcGFP). An example of an elav promoter is provided herein as SEQ ID NO: 8.

### DNA Constructs Expressing Human Amyloid Beta Isoforms in Zebrafish Neurons In Vivo.

Constructs comprise the zebrafish promoter for the neuron-specific gene elav, a Gal4/VP16-UAS construct to enhance transient expression of transgenes, and various isoforms of human Aβ or amyloid precursor protein (APP) fused to AcGFP.

Analyze Zebrafish Embryos Injected with the above DNA Constructs

Constructs are injected into embryos with red fluorescent neurons and analyzed under a fluorescent stereo microscope to determine whether fusion proteins are expressed and whether any change in fluorescent neurons can be detected. Immunohistochemistry can be performed to further characterize protein aggregates in the brain.

### DNA Constructs Expressing Human Tau Isoforms in Zebrafish Neurons In Vivo.

Constructs were made that link a zebrafish neuron-specific promoter to sequences encoding isoforms of human Tau in frame with a green fluorescent protein derived from Aequorea coerulescens (AcGFP), licensed from Clontech/BD Biosciences). Other fluorescent proteins could also be used as well as human proteins not fused to any fluorescent protein.

The promoter for the neuron-specific gene elav has been shown to successfully drive expression of enhanced green fluorescent protein (eGFP) in zebrafish neurons (Park et al., 2000). The zebrafish elav promoter has been cloned by this laboratory via PCR amplification from zebrafish genomic DNA. Applicants have also demonstrated transient expression of dsRed Express in neurons using this promoter. Other zebrafish promoters that could be used for this purpose include a nucleic acid comprising a gata-2 neuronal enhancer (Meng et al., 1997), and the alpha tubulin promoter. thy-1 is another neuron-specific promoters that can be utilized. An example of a nucleic acid comprising a GATA-2 promoter is set forth herein as SEQ ID NO: 10. Also provided is a nucleic acid comprising SEQ ID NO: 11 which corresponds to a neuron specific GATA-2 promoter.

Transient expression of transgenes in zebrafish is highly mosaic. With a neuron-specific promoter, only a subset of neurons will express the transgene in any given embryo. The level of expression may not be high enough to induce neuronal cell death. In addition, subtle signs of neuronal cell death may be difficult to visualize in the transgenic fish with green fluorescent neurons. To increase the level of transient expression, a Gal4/VP16 transcriptional activator coupled with a UAS promoter can be incorporated into DNA constructs (Köster and Fraser, 2001). Thus, a DNA fragment encoding GAL4/VP16:UAS (obtained from Reinhard Köster) can be optionally ligated into these constructs.

Human genes encoding isoforms of wild-type Tau can be obtained by PCR amplification from a pool of cDNA prepared from human brain (purchased from Clontech/BD Biosciences) and cloned into a TA cloning vector (Invitrogen). Three and four repeat forms of Tau can be identified by sequencing the cloned amplification products. The 3 repeat form of human Tau cats as a negative control, since this form does not form aggregates as easily as the 4 repeat form.

Mutations of interest can be obtained by site-directed mutagenesis (Stratagene) of the 4 repeat form of Tau. Briefly, primers of approximately 40 base pairs in length can be designed to be nearly identical to sequences in human Tau, but will contain point mutations that correspond to known mutations in human FTDP-17 (Hutton et al., 1998). Several mutations can be used for this purpose, as described below. Overexpression of the wild-type 4 repeat form of human Tau may mimic the effect of several FTDP-17 mutations that affect the 5′ splice site of exon 10 (Hutton et al., 1998). Polyacrylamide gel electrophoresis (PAGE)-purified primers can be purchased from Sigma.

The following constructs can be made:
(1) elav promoter-Gal4VP16-UAS-human Tau (3 repeat form) fused to AcGFP
(2) elav promoter-Gal4VP16-UAS-human Tau (4 repeat form) fused to AcGFP
(3) elav promoter-Gal4VP16-UAS-human Tau (4 repeat form) (P301L mutant) fused to AcGFP
(4) elav promoter-Gal4VP16-UAS-human Tau (4 repeat form) (R406W mutant) fused to AcGFP
(5) elav promoter-Gal4VP16-UAS-human Tau (G272V mutant) fused to AcGFP (for this construct, the 4 repeat form or the three repeat form of Tau with a G272V mutation can be utilized)
(6) elav promoter-Gal4VP16-UAS-human Tau (3 repeat form)
(7) elav promoter-Gal4VP16-UAS-human Tau (4 repeat form)
(8) elav promoter-Gal4VP16-UAS-human Tau (4 repeat form) (P301L mutant)
(9) elav promoter-Gal4VP16-UAS-human Tau (4 repeat form) (R406W mutant)
(10) elav promoter-Gal4VP16-UAS-human Tau (3 repeat form or 4 repeat form) (G272V mutant).

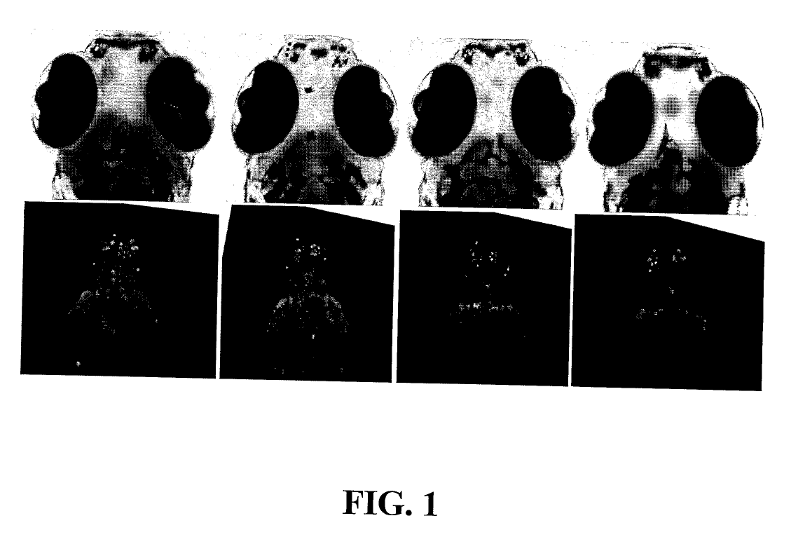

Data provided herein shows that overexpression of Tau-AcGFP fusion proteins causes a reduction in the fluorescence in the brain of transgenic embryos expressing red fluorescent protein in neurons (FIG. 1). Reduction in fluorescence was observed when constructs encoding isoforms of Tau that contain 4 microtubule binding domains were injected. Constructs encoding isoforms of Tau with only 3 microtubule domains appeared to have little effect on fluorescence. Furthermore, overexpression of the Tau-P301 mutant isoform had a dramatic effect on the survival of injected embryos, suggesting that it is pathogenic in zebrafish. All constructs were linearized prior to injection into zebrafish embryos at the one cell stage. Larvae were analyzed for fluorescence at 5 days post fertilization (dpf).

### DNA Constructs Expressing Human Amyloid Beta Isoforms in Zebrafish Neurons In Vivo.

DNA constructs can be designed using methodology similar to that described for part A. Constructs can be designed to express wild type and mutant forms of both the Aβ peptide and the full-length APP. Several point mutations in the Aβ peptide, which causes a familial form of AD, can be used. For example, the Arctic mutant peptide has been shown to aggregate more rapidly than wild-type Aβ and to be highly neurotoxic (Murakami et al., 2002). Aβ constructs will also include signal sequences to allow Aβ peptides to be secreted (Link, 1995). For APP, two different familial AD mutations (shown below) can be combined into one construct. Aβ constructs can include AcGFP sequences, but these constructs can also be made without AcGFP sequence. Because fusion of the small Aβ peptides with the much larger AcGFP molecule may impair aggregation, a construct without the AcGFP sequence is contemplated. If APP-AcGFP fusions are processed in the zebrafish brain in the same way as APP is processed in the human brain, the AcGFP will be fused to the C terminal portion of the protein. Thus, Aβ aggregates formed by overexpression of this protein will not be linked to a fluorescent marker.

The following constructs can be made to link the zebrafish elav promoter to Gal4/VP16-UAS sequences and sequences encoding either Aβ peptides or APP:

(1) elav promoter-Gal4VP16-UAS-signal sequence-human Aβ 42 peptide (wild-type) (the wild type human Aβ 42 nucleic acid encodes SEQ ID NO: 7)

(2) elav promoter-Gal4VP16-UAS-signal sequence-human Aβ 42 peptide, Arctic mutant (E22G)

The numbering of the mutations set forth herein correspond to the numbering of the wild type human Aβ (SEQ ID NO: 7). Therefore, E22G indicates that the glutamic acid at position 22 is mutated to glycine.

(3) elav promoter-Gal4VP16-UAS-signal sequence-human Aβ 42 peptide, Flemish mutant (A21G)

(4) elav promoter-Gal4VP16-UAS-signal sequence-human Aβ 42 peptide, Dutch mutant (E22Q)

(5) elav promoter-Gal4VP16-UAS-signal sequence-human Aβ 42 peptide, Italian mutant (E22K)

(6) elav promoter-Gal4VP16-UAS-signal sequence-human Aβ 42 peptide, Iowa mutant (D23N)

(7)) elav promoter-Gal4VP16-UAS-signal sequence-human Aβ 40 peptide (possible negative control)

An example of a signal sequence that can be utilized is set forth herein as SEQ ID NO: 9. However, one of skill in the art would know how to identify and utilize any signal sequence available in the art for the expression and secretion of a protein associated with Alzheimer's disease described herein.

DNA constructs expressing human amyloid beta isoforms in zebrafish neurons in vivo.

(1) elav promoter-Gal4VP16-UAS-human APP (wild-type) (for example, the human APP nucleic acid can encode SEQ ID NO: 3 or SEQ ID NO: 4).

(2) elav promoter-Gal4VP16-UAS-human APP (wild-type) fused to AcGFP.

(3) elav promoter-Gal4VP 16-UAS-human APP (K670N,M671L+V717F mutants)

(4) elav promoter-Gal4VP16-UAS-human APP (K670N,M671L+V717F mutants) fused to AcGFP.

Analyze Zebrafish Embryos Injected with the above DNA Constructs.

DNA constructs can be injected at the one cell stage into either wild-type embryos or transgenic embryos that express a red fluorescent protein (dsRed Express, Clontech) under the control of the elav promoter. Negative controls can include mock injections and the AcGFP vector. For Tau experiments, the Tau construct with three repeat domains can act as a negative control for the Tau constructs that contain four repeat domains. Following injections, embryos will be monitored under a fluorescent stereomicroscope over a period of several days. Observations under a GFP filter set allows observation of fusion proteins in the brain. Detection of neurofibrillary pathology may require observation of embryos using a confocal microscope.

Transgenic embryos injected with DNA constructs can be monitored with a rhodamine filter set to allow observation of potential neuronal cell death. However, transient expression is mosaic and may not produce high enough protein levels to induce neuronal cell death. Moreover, subtle damage to neurons may be difficult to visualize. It is possible that neuronal damage may be observed that does not involve neuronal cell loss. For example, enlarged axonal and dendritic varicosities associated with Aβ deposits can be observed. Fluorescent neurons in the zebrafish model can be observed for abnormal morphology as well as degeneration. Embryos can also be fixed and sectioned to allow higher resolution imaging of neuronal morphology.

Another possible mechanism for visualization of neuronal damage is upregulation of the astrocyte-specific marker glial fibrillary acidic protein (GFAP). A transgenic fish expressing fluorescent protein under the control of the GFAP promoter could be used to measure damage induced by Aβ or Tau overexpression. Zebrafish GFAP has been cloned and shown to be 67% identical to human GFAP (Nielsen et al., 2003). Fluorescent probes for caspase activation, nuclear shrinkage (Hoechst staining) and/or other death gene activation pathway markers can be used as alternative readouts for neurodegeneration. Fluorojade, a stain specific for neurodegeneration, could also be used to detect neuron cell death.

Wild-type embryos injected with DNA constructs can be prepared for whole mount immunohistochemistry. Antibodies to human Aβ or Tau can be used to monitor expression of protein in the brain and can be used to detect protein aggregation, plaques, and tangles in transgenic zebrafish. The Congo red and Thioflavin S dyes can also be tested to determine whether they can be used to detect Aβ aggregates in the zebrafish brain.

Embryos transiently expressing fusion proteins will be raised to adulthood to identify stable founders. High levels of transient expression may be lethal to larvae and prevent efficient creation of stable transgenic lines. However, an inducible system can be utilized to circumvent this problem. The ability to temporally regulate expression is also useful. For example, it has been shown that when Gal4 is fused to a portion of the glucocorticoid receptor, transgenes driven by the UAS promoter can be activated by application of dexamethasone (de Graaf et al., 1998). It is possible that a Gal4-glucocorticoid receptor fusion protein could be driven by a neuron-specific promoter to combine tissue specificity with precise temporal regulation.

The mechanism of neuronal cell death in AD is still controversial. If aggregation of Aβ or Tau inclusions is not sufficient for neuronal cell death, alternative constructs can be made, such as a combination of mutant Tau and APP or Aβ. If aggregates of Aβ are not observed in transgenic animals overexpressing Aβ or APP, transgenic expression of a zinc transporter can be included, since concentration of zinc in the brain has been shown to play a role in Aβ aggregation (Bush, 2003).

### Target Validation Using Zebrafish AD Models

Genes can be tested for their role in tangle or aggregate formation and/or neuroprotection in zebrafish. Zebrafish orthologues of human genes of interest can be identified and antisense molecules, such as morpholinos (Nasevicius et al., 2000; GeneTools, Inc.) or gripNAs (Urtishak et al., 2003; Active Motif), can be designed to target the 5′ untranslated region, translational start site or alternative splice site of those genes. Transgenic AD model embryos can be injected with antisense molecules at the single-cell stage. Embryos will be allowed to develop until the time of the assay (i.e., when aggregates are known to form). An antisense molecule that increases the number of neurons or decreases the formation of fibrillary tangles or aggregates will be considered neuroprotective for AD. If antisense molecules targeting alternative splice sites are used, the level of knockdown can be assessed via RT-PCR.

Zebrafish AD models can also be used for forward genetic screens to identify novel genes involved in plaque or tangle formation and to identify potential targets for AD therapy.

### Automation and Compound Screening

Fluorescence-based zebrafish AD assays can be automated, making them amenable to compound screening and large scale antisense knockdown. For example, the Discovery-1™ high content screening system (Molecular Devices) can be utilized to automatically capture images and quantify the data for transgenic fluorescent zebrafish assays. Either Discovery-1 or other screening systems, such as the Opera screening system (Evotec OAI) which has laser confocal capability and faster motorized objectives, can be used to automate the AD assays.

To increase throughput, transgenic AD model embryos can be arrayed into 96- or 384-well plates in the absence or presence of test compounds. The duration of compound treatment will depend on the time required for formation of neurofibrillary tangles or Aβ aggregates and/or neurodegeneration. Plates will be scanned on Discovery-1 using 1×, 2×, 4×, 10×, 20× and 40× objectives and alternating filters to detect GFP, DsRed Express, fluorescent secondary antibodies, or fluorescent probes for caspase activation. Z-series acquisition may be needed to resolve different planes of neuronal fluorescence. Fluorescence intensity and distribution will be measured to assess tangle or aggregate formation or neuronal cell death. Compound-induced changes in tangle or aggregate formation and/or neuroprotection will be evaluated by comparing AD model embryos in the absence and presence of test compounds. For instance, a decrease in tangle or aggregate formation in the presence of a test compound would indicate that the compound can prevent aggregate formation in AD. Alternatively, an increase in the number of neurons in the presence of a test compound can indicate neuroprotective activity. Other indicators of neuron morphology can also be used.

Throughout this application, various publications are referenced. The disclosures of these publications in their entireties are hereby incorporated by reference into this application in order to more fully describe the state of the art to which this invention pertains.

### REFERENCES

Babin P J, Thisse C, Durliat M, Andre M, Akimenko M A, Thisse B. (1997). Both apolipoprotein E and A-I genes are present in a nonmammalian vertebrate and are highly expressed during embryonic development. Proc Natl Acad Sci USA. 94:8622-8627.
Buée L, Bussière T, Buée-Scherrer V, Delacourte A, H of P R. (2000). Tau protein isoforms, phosphorylation and role in neurodegenerative disorders. Brain Res Rev 95-130.
Brendza RP, O'Brien C, Simmons K, McKeel D W, Bales K R, Paul S M, Olney J W, Sanes J R, Holtzman D M. (2003). PDAPP;YFP double transgenic mice: a tool to study amyloid-β associated changes in axonal, dendritic, and synaptic structures. J. Comp. Neurol. 456, 275-383.
Bush A I. (2003). The metallobiology of Alzheimer's disease. Trends Neurosci. 26, 207-214.
Chishti M A, Yang D S, Janus C, Phinney A L, Home P, Pearson J, Strome R, Zuker N, Loukides J, French J, Turner S, Lozza G, Grilli M, Kunicki S, Morissette C, Paquette J, Gervais F, Bergeron C, Fraser P E, Carlson G A, George-Hyslop P S, Westaway D. (2001). Early-onset amyloid deposition and cognitive deficits in transgenic mice expressing a double mutant form of amyloid precursor protein 695. J Biol Chem. 276:21562-21570.
Fan L, Crodian J, Collodi P. (2004). Culture of embryonic stem cell lines from zebrafish. Methods Cell Biol. 76, 151-160.

Goldman D, Hankin M, Li Z, Dai X, Ding J. (2001). Transgenic zebrafish for studying nervous system development and regeneration. Transgenic Res. 10, 21-33. de Graaf M, Zivkovic D, Joore J. (1998). Hormone-inducible expression of secreted factors in zebrafish embryos. Develop. Growth Differ. 40: 577-582.
Groth C, Nomes S, McCarty R, Tamme R, Lardelli M. (2002). Identification of a second presenilin gene in zebrafish with similarity to the human Alzheimer's disease gene presenilin. Dev Genes Evol. 212:486-490.
Gúenette S Y, Tanzi R E. (1999). Progress toward valid transgenic mouse models for Alzheimer's disease. Neurobiol. Aging 20, 201-211.
Guo S. (2004). Linking genes to brain, behavior and neurological diseases: what can we learn from zebrafish? Genes Brain Behav. 3, 63-74.
Higashijima S, Hotta Y, Okamoto H. (2000). Visualization of cranial motor neurons in live transgenic zebrafish expressing green fluorescent protein under the control of the islet-1 promoter/enhancer. J. Neurosci. 20, 206-218.
Hsiao K, Chapman P, Nilsen S, Eckman C, Harigaya Y, Younkin S, Yang F, Cole G. (1996). Correlative memory deficits, Aβ elevation, and amyloid plaques in transgenic mice. Science 274, 99-102.

Hutton M, Lendon C L, Rizzu P, Baker M, Froelich S, Houlden H, Pickering-Brown S, Chakraverty S, Isaacs A, Grover A, Hackett J, Adamson J, Lincoln S, Dickson D, Davies P, Petersen R C, Stevens M, de Graaff E, Wauters E, van Baren J, Hillebrand M, Joosse M, Kwon J M, Nowotny P, Heutink P, et al. (1998). Association of missense and 5′ splice-site mutations in tau with the inherited dementia FTDP-17. Nature 393: 702-705.
Klein W L, Krafft G A, Finch C E. (2001). Targeting small Aβ oligomers: the solution to an Alzheimer's disease conundrum? Trends Neurosci. 24, 219-224.
Koster R W, Fraser S E. (2001). Tracing transgene expression in living zebrafish embryos. Dev Biol. 233:329-346.
Kraemer B C, Zhang B, Leverenz J B, Thomas J H, Trojanowski J Q, Schellenberg G D. (2003). Neurodegeneration and defective neurotransmission in a Caenorhabditis elegans model of tauopathy. Proc. Natl. Acad. Sci. USA 100, 9980-9985.

Leimer U, Lun K, Romig H, Walter J, Grunberg J, Brand M, Haass C. (1999). Zebrafish (Danio rerio) presenilin promotes aberrant amyloid beta-peptide production and requires a critical aspartate residue for its function in amyloidogenesis. Biochemistry. 38, 13602-13609.
Lewis J, Dickson D W, Lin W L, Chishohn L, Corral A, Jones G, Yen S H, Sahara N, Skipper L, Yager D, Eckman C, Hardy J, Hutton M, McGowan E. (2003). Enhanced neurofibrillary degeneration in transgenic mice expressing mutant Tau and APP. Science 293, 1487-1491.
Link C D. (1995). Expression of human β-amyloid peptide in transgenic Caenorhabditis elegans. Proc. Natl. Acad. Sci. USA 92, 9368-9372.
Meng, A, Tang H, Ong B A, Farrell M J and Lin S. (1997). Promoter analysis in living zebrafish embryos identifies a cis-acting motif required for neuronal expression of GATA-2. Proc. Natl. Acad. Sci. USA 94, 6267-6272.
Musa A, Lehrach H; Russo V A. (2001). Distinct expression patterns of two zebrafish homologues of the human APP gene during embryonic development. Dev Genes Evol. 211:563-567.
Nasevicius A, Ekker S C. (2000). Effective targeted gene ‘knockdown’ in zebrafish. Nat Genet. 26:216-220.
Neuhauss S C. (2003). Behavioral genetic approaches to visual system development and function in zebrafish. J. Neurobiol. 54, 148-160.
Nielsen A L, Jorgensen A L. (2003). Structural and functional characterization of the zebrafish gene for glial fibrillary acidic protein, GFAP. Gene 310, 123-132.
Nornes S, Groth C, Camp E, Ey P, Lardelli M. Developmental control of Presenilin1 expression, endoproteolysis, and interaction in zebrafish embryos. (2003). Exp Cell Res. 289:124-132.
Oehlmann V D, Berger S, Sterner C, Korsching S I. (2004). Zebrafish beta tubulin 1 expression is limited to the nervous system throughout development, and in the adult brain is restricted to a subset of proliferative regions. Gene Expr Patterns 4, 191-198.
Park H C, Kim C H, Bae Y K, Yeo S Y, Kim S H, Hong S K, Shin J, Yoo K W, Hibi M, Hirano T, Miki N, Chitnis A B, Huh T L. (2000). Analysis of upstream elements in the HuC promoter leads to the establishment of transgenic zebrafish with fluorescent neurons. Dev Biol. 227, 279-293.

Petit A, Pasini A, Alves Da Costa C, Ayral E, Hernandez J F, Dumanchin-Njock C, Phiel C J, Marambaud P, Wilk S, Farzan M, Fulcrand P, Martinez J, Andrau D, Checker F. (2003). JLK isocoumarin inhibitors: Selective gamma-secretase inhibitors that do not interfere with notch pathway in vitro or in vivo. J Neurosci Res. 74:370-377.
Racchi M, Govoni S. (2003). The pharmacology of amyloid precursor protein processing. Exp. Gerontol. 38, 145-157.
Rubinstein A L. (2003). Zebrafish: from disease modeling to drug discovery. Curr Opin Drug Discov Devel 6:218-223.
Saint-Amant, L and Drapeau, P. (1998). Time course of the development of motor behaviors in the zebrafish embryo. J. Neurobiol. 37(4): 622-32.
Styren S D, Hamilton R L, Sytren G C, Klunk S W (2000). X-34, A Fluorescent Derivative of Congo Red: A Novel Histochemical Stain for Alzheimer's Disease Pathology. Journal of Histochemistry and Cytochemistry. 48:1223-1232.
Sun A, Nguyen S V, Bing G (2002) Comparative analysis of an improved thioflavin-S staing, Gallyas silver stain, and immunhistochemistry for neurofibrillary tangle demonstration on the same sections. Journal of Histochemistry and Cytochemistry. 50:463-472.
Tandon A, Fraser P. (2002). The presenilins. Genome Biol. 3(11): reviews.3014.1-3014.9.
Tomasiewicz H G, Flaherty D B, Soria J P, Wood J G. (2002). Transgenic zebrafish model of neurodegeneration. J Neurosci Res. 70:734-745.
Urtishak K A, Choob M, Tian X, Steruheim N, Talbot W S, Wickstrom E, Farber S A. (2003). Targeted gene knockdown in zebrafish using negatively charged peptide nucleic acid mimics. Dev Dyn. 228:405-413.
Verdile G, Fuller S, Atwood C S, Laws S M, Gandy S E, Martins R N. (2004). The role of beta amyloid in Alzheimer's disease: still a cause of everything or the only one who got caught? Pharmacol. Res. 50.397-409.
Williams F E, White D, Messer W S. (2002). A simple spatial alternation task for assessing memory function in zebrafish. Behav Processes 58, 125-132.
Wittmann C W, Wszolek M F, Shulman J M, Salvaterra P M, Lewis J, Hutton M, Feany M B. (2001). Tauopathy in Drosophila: Neurodegeneration without neurofibrillary tangles. Science 293, 711-714.

1. A transgenic zebrafish that expresses a Tau polypeptide, comprising a zebrafish neuron specific expression sequence operably linked to a nucleic acid sequence encoding a Tau polypeptide wherein the Tau polypeptide is expressed in the neurons of the transgenic zebrafish, and wherein the transgenic zebrafish exhibits a pathology associated with Alzheimer's Disease.

2. The transgenic zebrafish of claim 1 further comprising a zebrafish neuron specific expression sequence operably linked to a nucleic acid sequence encoding a fluorescent reporter polypeptide.

3. The transgenic zebrafish of claim 2, wherein the fluorescent reporter polypeptide is selected from the group consisting of GFP, AcGFP and DsRedExpress.

4. The transgenic zebrafish of claim 1, wherein the neuron specific expression sequence is a neuron-specific promoter.

5. The transgenic zebrafish of claim 4, wherein the neuron-specific promoter is selected from the group consisting of an elav promoter and a GATA-2 promoter.

6. The transgenic zebrafish of claim 1, wherein the zebrafish neuron specific expression sequence and the sequence encoding the Tau polypeptide are contained in an exogenous construct.

7. (canceled)

8. (canceled)

9. (canceled)

10. The transgenic zebrafish of claim 1, wherein the expression sequence comprises an inducible promoter.

11. The transgenic zebrafish of claim 10, wherein the inducible promoter is an inducible UAS promoter activated by GAL4/VP16.

12. (canceled)

13. A transgenic zebrafish that expresses a Tau fusion polypeptide, comprising a zebrafish neuron specific expression sequence operably linked to a nucleic acid sequence encoding a fusion polypeptide comprising a Tau polypeptide and a fluorescent reporter polypeptide, wherein the fusion polypeptide is expressed in the neurons of the transgenic zebrafish, and wherein the transgenic zebrafish exhibits a pathology associated with Alzheimer's Disease.

14. The transgenic zebrafish of claim 13, wherein the fluorescent reporter polypeptide is selected from the group consisting of GFP, AcGFP and DsRedExpress.

15. The transgenic zebrafish of claim 13 further comprising a zebrafish neuron specific expression sequence operably linked to a nucleic acid sequence encoding a fluorescent reporter polypeptide that is different from the reporter polypeptide fused to the Tau polypeptide.

16. The transgenic zebrafish of claim 13, wherein the neuron specific expression sequence is a neuron-specific promoter.

17. The transgenic zebrafish of claim 15, wherein the neuron-specific promoter is selected from the group consisting of an elav promoter and a GATA-2 promoter.

18. The transgenic zebrafish of claim 13, wherein the zebrafish neuron specific expression sequence and the sequence encoding the fusion polypeptide are contained in an exogenous construct.

19. (canceled)

20. (canceled)

21. (canceled)

22. The transgenic zebrafish of claim 13, wherein the expression sequence comprises an inducible promoter.

23. The transgenic zebrafish of claim 22, wherein the inducible promoter is an inducible UAS promoter activated by GAL4/VP16.

24. (canceled)

25. A transgenic zebrafish that expresses an APP polypeptide, comprising a zebrafish neuron specific expression sequence operably linked to a nucleic acid sequence encoding an APP polypeptide wherein the APP polypeptide is expressed in the neurons of the transgenic zebrafish, and wherein the transgenic zebrafish exhibits a pathology associated with Alzheimer's Disease.

26. The transgenic zebrafish of claim 25 further comprising a zebrafish neuron specific expression sequence operably linked to a nucleic acid sequence encoding a fluorescent reporter polypeptide.

27. The transgenic zebrafish of claim 26, wherein the fluorescent reporter polypeptide is selected from the group consisting of GFP, AcGFP and DsRedExpress.

28. The transgenic zebrafish of claim 25, wherein the neuron specific expression sequence is a neuron-specific promoter.

29. The transgenic zebrafish of claim 28, wherein the neuron-specific promoter is selected from the group consisting of an elav promoter and a GATA-2 promoter.

30. The transgenic zebrafish of claim 25, wherein the zebrafish neuron specific expression sequence and the sequence encoding the APP polypeptide are contained in an exogenous construct.

31. (canceled)

32. (canceled)

33. (canceled)

34. (canceled)

35. The transgenic zebrafish of claim 25, wherein the expression sequence comprises an inducible promoter.

36. The transgenic zebrafish of claim 35, wherein the inducible promoter is an inducible UAS promoter activated by GAL4/VP16.

37. (canceled)

38. A transgenic zebrafish that expresses an APP fusion polypeptide comprising a zebrafish neuron specific expression sequence operably linked to a nucleic acid sequence encoding a fusion polypeptide comprising an APP polypeptide and a fluorescent reporter polypeptide, wherein the fusion polypeptide is expressed in the neurons of the transgenic zebrafish, and wherein the transgenic zebrafish exhibits a pathology associated with Alzheimer's disease.

39. The transgenic zebrafish of claim 38, wherein the fluorescent reporter polypeptide is selected from the group consisting of GFP, AcGFP and DsRedExpress.

40. The transgenic zebrafish of claim 38 further comprising a zebrafish neuron specific expression sequence operably linked to a nucleic acid sequence encoding a fluorescent reporter polypeptide that is different from the reporter polypeptide fused to the APP polypeptide.

41. The transgenic zebrafish of claim 38, wherein the neuron specific expression sequence is a neuron-specific promoter.

42. The transgenic zebrafish of claim 41, wherein the neuron-specific promoter is selected from the group consisting of an elav promoter and a GATA-2 promoter.

43. The transgenic zebrafish of claim 38, wherein the neuron specific expression sequence and the sequence encoding the fusion polypeptide are contained in an exogenous construct.

44. (canceled)

45. (canceled)

46. (canceled)

47. (canceled)

48. The transgenic zebrafish of claim 38, wherein the expression sequence comprises an inducible promoter.

49. The transgenic zebrafish of claim 48, wherein the inducible promoter is an inducible UAS promoter activated by GAL4/VP16.

50. (canceled)

51. A transgenic zebrafish that expresses an amyloid P polypeptide comprising comprising a neuron specific expression sequence operably linked to a nucleic acid sequence encoding an amyloid β polypeptide wherein the amyloid β is expressed in the neurons of the transgenic zebrafish, and wherein the transgenic zebrafish exhibits a pathology associated with Alzheimer's disease.

52. The transgenic zebrafish of claim 51, further comprising a neuron specific expression sequence operably linked to a nucleic acid sequence encoding a fluorescent reporter polypeptide.

53. The transgenic zebrafish of claim 51, wherein the neuron specific expression sequence is a neuron-specific promoter.

54. The transgenic zebrafish of claim 53, wherein the neuron-specific promoter is selected from an elav promoter or a GATA-2 promoter.

55. The transgenic zebrafish of claim 51, wherein the neuron specific expression sequence and the sequence encoding the amyloid β polypeptide are contained in an exogenous construct.

56. (canceled)

57. (canceled)

58. (canceled)

59. (canceled)

60. (canceled)

61. The transgenic zebrafish of claim 51, wherein the expression sequence comprises an inducible promoter.

62. The transgenic zebrafish of claim 51, wherein the inducible promoter is an inducible UAS promoter activated by GAL4/VP16.

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. The transgenic zebrafish of claim 13, further comprising a zebrafish neuron specific expression sequence operably linked to a nucleic acid sequence encoding an APP polypeptide wherein the APP polypeptide is expressed in the neurons of the transgenic zebrafish, and wherein the transgenic zebrafish exhibits a pathology associated with Alzheimer's disease.

91. The transgenic zebrafish of claim 90, further comprising a zebrafish neuron specific expression sequence operably linked to a nucleic acid sequence encoding a fluorescent reporter polypeptide that is different from the fluorescent reporter polypeptide fused to Tau.

92. The transgenic zebrafish of claim 13, further comprising a zebrafish neuron specific expression sequence operably linked to a nucleic acid sequence encoding a fusion polypeptide comprising an APP polypeptide and a fluorescent reporter polypeptide, wherein the APP fusion polypeptide is expressed in the neurons of the transgenic zebrafish, and wherein the transgenic zebrafish exhibits a pathology associated with Alzheimer's disease.

93. The transgenic zebrafish of claim 92, further comprising a zebrafish neuron specific expression sequence operably linked to a nucleic acid sequence encoding a fluorescent reporter polypeptide that is different from the reporter polypeptide fused to the Tau polypeptide and different from the reporter polypeptide fused to the APP polypeptide.

94. A method of identifying an agent that modulates a pathology associated with Alzheimer's disease comprising:
a) contacting the zebrafish of claim 1, 2, 13 or 15 with a test agent;
b) comparing the neuronal pathology of the zebrafish contacted with the test agent to the neuronal pathology of a zebrafish of claim 1, 2 13 or 15 not contacted with the test agent;
c) determining the effect of the test agent on the zebrafish, such that if there is a difference in the neuronal pathology of the zebrafish contacted with the test agent and the zebrafish not contacted with the test agent, the test agent is an agent that modulates a pathology associated with Alzheimer's disease.

95. The method of claim 94, wherein the difference in neuronal pathology is a decrease in neuronal cell death, a decrease in neurofibrillary tangles, a decrease in neuronal fluorescence, or a decrease in Tau expression in the zebrafish contacted with the test agent as compared to the zebrafish not contacted with the test agent.

96. (canceled)

97. (canceled)

98. (canceled)

99. A method of identifying an agent that modulates a pathology associated with Alzheimer's disease comprising:
a) contacting the zebrafish of claim 25, 26, 38 or 40 with a test agent;
b) comparing the neuronal pathology of the zebrafish contacted with the test agent to the neuronal pathology of a zebrafish of claim 25, 26, 38 or 40 not contacted with the test agent;
c) determining the effect of the test agent on the zebrafish, such that if there is a difference in the neuronal pathology pathology of the zebrafish contacted with the test agent and the zebrafish not contacted with the test agent, the test agent is an agent that modulates a pathology associated with Alzheimer's disease.

100. The method of claim 99, wherein the difference in neuronal pathology is a decrease in neuronal cell death, a decrease in neurofibrillary tangles, a decrease in neuritic plaques, a decrease in neuronal fluorescence or a decrease in APP expression in the zebrafish contacted with the test agent as compared to the zebrafish not contacted with the test agent.

101. (canceled)

102. (canceled)

103. (canceled)

104. (canceled)

105. A method of identifying an agent that modulates a pathology associated with Alzheimer's disease comprising:
a) contacting the zebrafish of claim 51 or claim 52 with a test agent;
b) comparing the neuronal pathology of the zebrafish contacted with the test agent to the neuronal pathology of a zebrafish of claim 51 or claim 52 not contacted with the test agent;
c) determining the effect of the test agent on the zebrafish, such that if there is a difference in the neuronal pathology of the zebrafish contacted with the test agent and the zebrafish not contacted with the test agent, the test agent is an agent that modulates a pathology associated with Alzheimer's disease.

106. The method of claim 105, wherein the difference in neuronal pathology is a decrease in neuronal cell death, a decrease in neurofibrillary tangles, a decrease in neuritic plaques, a decrease in neuronal fluorescence or a decrease in amyloid β expression in the zebrafish contacted with the test agent as compared to the zebrafish not contacted with the test agent.

107. (canceled)

108. (canceled)

109. (canceled)

110. (canceled)

111. (canceled)

112. (canceled)

113. (canceled)

114. (canceled)

115. (canceled)

116. (canceled)

117. A method of identifying an agent that modulates a pathology associated with Alzheimer's disease comprising:
a) contacting the zebrafish of claim 90, 91, 92 or 93 with a test agent;
b) comparing the neuronal pathology of the zebrafish contacted with the test agent to the neuronal pathology of a zebrafish of claim 90, 91, 92 or 93 not contacted with the test agent;
c) determining the effect of the test agent on the zebrafish, such that if there is a difference in the neuronal pathology pathology of the zebrafish contacted with the test agent and the zebrafish not contacted with the test agent, the test agent is an agent that modulates a pathology associated with Alzheimer's disease.

118. The method of claim 117, wherein the difference in neuronal pathology is a decrease in neuronal cell death, a decrease in neurofibrillary tangles, a decrease in neuritic plagues, a decrease in neuronal fluorescence, a decrease in APP expression or a decrease in Tau expression in the zebrafish contacted with the test agent as compared to the zebrafish not contacted with the test agent.

119. (canceled)

120. (canceled)

121. (canceled)

122. (canceled)

123. (canceled)

124. (canceled)

125. (canceled)

126. (canceled)

127. (canceled)
